(19) United States
(12) Patent Application Publication
(10) Pub. No.: US 2007/0003753 A1
(43) Pub. Date: Jan. 4, 2007

(54) Medical devices comprising a reticulated composite material

(76) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(21) Appl. No.: 11/479,658

(22) Filed: Jun. 30, 2006

Related U.S. Application Data
(60) Provisional application No. 60/696,255, filed on Jul. 1, 2005.

(51) Int. Cl. A61L 27/44

(52) U.S. Cl. 428/315.5; 623/926

(57) ABSTRACT

The present invention relates to medical devices, particularly for therapeutic and/or diagnostic purposes, which may coated or at least partially formed using porous reticulated composite materials. Specifically, the present invention relates to medical devices which include a porous composite material, where the composite material can be formed using a process comprising the steps of providing a liquid mixture that includes at least one inorganic and/or organic reticulating agent; and at least one matrix material that is a polymer or combination of polymers; and solidifying the liquid mixture.

### CROSS-REFERENCE TO RELATED APPLICATION(S)

This application claims priority from U.S. Patent Application No. 60/696,255, filed Jul. 1, 2005, the entire disclosure of which is incorporated herein by reference.

### FIELD OF THE INVENTION

The present invention relates generally to medical devices, including devices which may be used for therapeutic and/or diagnostic purposes, comprising porous reticulated composite materials and methods for the production thereof. The present invention further relates to one or more medical devices comprising a porous composite material which may be produced by providing a liquid mixture which includes at least one inorganic and/or organic reticulating agent and at least one matrix material that may be a polymer or a polymer mixture, and solidifying the liquid mixture.

### BACKGROUND INFORMATION

Porous materials may play an increasingly important role in biomedical applications for use as implantable materials, drug carriers, etc. The use of composite materials can provide a combination of components having different physico-chemical properties, resulting in a composite material which may have new or improved properties. Composite materials may exhibit a similar or superior stability, biocompatibility and/or strength at less overall weight when compared to non-composite materials.

Porous composite materials may be prepared using conventional sintering methods. For example, powders that may include fibers, dendritic or spherically-formed precursor particles can be pressed into molds or extruded and then subjected to a sinter process. In such materials, the rigidity of the material, the pore size and/or the surface area may depend on the packaging density as well as on the size, form and/or the composition of particles in the powders.

One potential disadvantage of conventional sintering methods may be that the adjustment of pore sizes can be difficult to control, and the mechanical properties may not be sufficiently tailored, for example, with respect to pore size, porosity or specific surface area. Parameters used in a sintering process may also have an influence on the strength, pore size and surface area of the porous materials. Pore sizes can be adjusted in additional processing steps, e.g. by deposition from the gas phase, electroplating or electroless plating, which may decrease the size of large pores by adding further material while providing a more homogeneous pore size distribution. These techniques, however, can reduce the available surface in porous materials. Other techniques that may be used can include spray-coating of pre-sintered porous materials with a slurry, subsequent drying, and repeated sintering. These techniques may provide a pore diffusion of material from the slurry into the porous sintered structured, and to insufficient adhesion of the material deposited in the procedure, which can result from different thermal coefficients of expansion and shrinking of the deposited material.

International Patent Publication WO 04/054625 describes a technique in which a pre-sintered porous material is coated by powdered nanoparticle material and subsequently re-sintered. International Patent Publication WO 99/15292 describes a process in which porous fiber-containing composite structures may be obtained from a dispersion of fibers with the use of binders and subsequent gasification of the mixture prior to, during or after sintering.

A further potential disadvantage of the above-described methods may be that the sintering processes are conventionally performed at high temperatures, which can have undesirable effects on materials that are used, e.g., for coating of medical devices that may not have sufficient thermal stability. For example, stents made of shape-memory alloys or artificial heart valves made of polymeric materials can be sensitive to extreme temperatures. Another potential disadvantage of these methods may be that the material is processed in costly molding processes into a stable two- or three-dimensional structure, and only restricted forms may be obtainable due to the brittleness of the materials.

Conventional processing of porous materials can also require several post-treatment processing steps, and the sintering process may likely be applicable only to inorganic composites because of necessary process conditions.

Thus there may be a need for providing porous coatings on medical devices having improved properties, and coating materials which have physico-chemical properties, such as biocompatibility, that may be adapted to specific application requirements. Furthermore, there may be a need to functionalize a porous coating on medical devices or the material of the device itself, e.g. to impart signaling properties allowing for improved detection of such coated devices by imaging methods. There may also be a need for providing medical devices comprising functional porous materials which may be produced in a cost efficient manner, and a process for manufacture thereof.

### SUMMARY OF EXEMPLARY EMBODIMENTS OF THE INVENTION

Therefore, it is one object of the present invention to provide a functionally coated medical device, the coating of which may be based on organic and/or inorganic particles in combination with suitable matrix materials and which has properties which may be easily modifiable.

A further object of the present invention is to provide improved medical devices comprising a material having properties which may be individually tailored to the specific application of the device.

A further object of the invention is to provide adjustable network-like structural properties in a material which may be self-organizing, and to produce coatings that may be structured in two or three dimensions. It is a further object of the present invention to provide a fine structuring in materials such as, e.g., adjustment of porosity, which may be achieved without deteriorating the chemical and/or physical stability of the material.

Yet another object of the present invention is to provide a medical device comprising a material that may be used as a coating as well as a bulk material, where the material has specific physico-chemical properties.

A still further object of the present invention is to provide a medical device which may be produced entirely or partially from a functional porous composite material having specific and/or preselected desired properties.

Yet a further object of the present invention is to provide a method for the production of porous reticulated composite materials from inexpensive starting materials which may have broadly variable properties, in a cost-efficient manner and using relatively few process steps.

Another object of the present invention is to provide a method for manufacturing medical devices and/or coatings on such devices which can be made from porous composite materials, where the properties of the material, e.g., biocompatibility, thermal coefficient of expansion, electric, dielectric, conductive or semi-conductive and magnetic or optical properties and any combinations thereof can be adjusted.

These and other objects of the invention can be achieved by exemplary embodiments of the present invention which may provide a medical device comprising a porous composite material. For example, the composite material can include one or more reticulating agents and at least one matrix material, and the matrix material may include at least one organic polymer. The reticulating agent may be embedded in the matrix material.

In a further exemplary embodiment of the present invention, a medical device as described above may be provided. For example, the composite material can be formed by a process that includes the steps of providing a liquid mixture that includes at least one reticulating agent and at least one matrix material that includes at least one organic polymer, and solidifying the mixture.

In a still further exemplary embodiment of the invention, a medical device is provided that can include a coating comprising a porous composite material. For example, the composite material comprises at least one reticulating agent and at least one matrix material, and wherein the matrix material includes at least one organic polymer. A medical device provided in accordance with certain exemplary embodiments of the present invention may be formed, partially or substantially entirely, from such a composite material and/or it may have a coating comprising such a composite material, where the coating may cover at least a portion of the surface of the device

In a further exemplary embodiment of the present invention, the porous composite material may have a porous reticulated structure, with pore sizes ranging from about 1 nm to about 400 micrometers. In another exemplary embodiment of the present invention, the porous composite material may have pore sizes ranging from about 500 mm to about 1000 micrometers.

In a still further exemplary embodiment of the present invention, the composite material may include one or more reticulating agents provided in the form of particles, such as nano- or micro-crystalline particles. The reticulating agents may comprise at least two particle size fractions of the same or different materials, where the fractions differ in size by a factor of at least 1.1, or at least 2.

In another exemplary embodiment of the present invention, the reticulating agent included in the composite material may be provided in the form of tubes, fibers or wires.

In further exemplary embodiments of the present invention, the reticulating agents included in the composite materials and devices made therefrom, as described above, may comprise inorganic materials such as, for example, metals, metal compounds, metal oxides, semiconductive metal compounds, and/or carbon species such as carbon fiber, graphite, soot, carbon black, fullerenes, or nanotubes. The reticulating materials may include particulate organic materials or fibers made of organic materials such as polymers, oligomers or pre-polymers, for example, a synthetic homopolymer or copolymer of an aliphatic or aromatic polyolefin, such as polyethylene or polypropylene; or a biopolymer.

In still further exemplary embodiments of the present invention, the reticulating agents included in devices as described above may comprise at least one inorganic material in combination with at least one organic material, or a combination of at least one particulate material with at least one material having a form selected from tubes, fibers or wires.

In further exemplary embodiments of the invention, the matrix materials included in the composite materials and devices made therefrom, as described above, may include oligomers, polymers, copolymers or prepolymers, thermosets, thermoplastics, synthetic rubbers, extrudable polymers, injection molding polymers, or moldable polymers such as, for example, epoxy resins, phenoxy resins, alkyd resins, epoxy-polymers, poly(meth)acrylate, unsaturated polyesters, saturated polyesters, polyolefines, rubber lattices, polyamides, polycarbonates, polystyrene, polyphenol, polysilicone, polyacetale, cellulose, or cellulose derivatives.

In still further exemplary embodiments of the present invention, a medical device as described above can be provided in the form of implants suitable for insertion into the human or animal body, for example, medical devices or implants for therapeutic or diagnostic purposes, which may include vascular endoprostheses, stents, coronary stents, peripheral stents, surgical implants, orthopedic implants, orthopedic bone prosthesis, joint prosthesis, bone substitutes, vertebral substitutes in the thoracic or lumbar region of the spinal column; artificial hearts, artificial heart valves, subcutaneous implants, intramuscular implants, implantable drug-delivery devices, catheters, guide wires for catheters or parts thereof, surgical instruments, surgical needles, screws, nails, clips, staples, support for cultivation of living material or scaffolds for tissue engineering. The medical device may be provided in the form of, for example, a stent, a drug eluting stent, a drug delivery implant, or a drug eluting orthopedic implant. Any of the medical devices above may include implants comprising signaling agents, markers, or therapeutically active agents.

In yet further exemplary embodiments of the present invention, the devices as described above can include active agents, which may be controllably releasable from the device. The active agents may include, for example, biologically active agents such as microorganisms, viral vectors, cells or living tissue, therapeutically active agents which can be resolvable or extractable from the composite material in the presence of physiologic fluids. The agents may also be used for diagnostic purposes such as, for example, a marker, a contrast medium or a radiopaque material which can be detectable by, or which can produce a signal detectable by, physical, chemical or biological detection methods, e.g., x-rays, nuclear magnetic resonance (NMR), computer tomography methods, scintigraphy, single-photon-emission computed tomography (SPECT), ultrasonic, radiofrequency (RF), or optical coherence tomography (OCT).

In further exemplary embodiments of the present invention, reticulating agents included in the devices and/or composite materials as described above may be capable of forming a network-like structure and/or self-orienting into a three dimensional structure.

In further exemplary embodiments of the invention, the composite material may include a reticulating agent such as, for example, soot, fullerenes, carbon fibers, silica, titanium dioxide, metal particles, tantalum particles, or polyethylene particles; and the matrix material may comprise epoxy resins or phenoxy resins. A composite material used to form a device or a portion thereof, or a coating thereon, may be obtained from a liquid mixture comprising at least one organic solvent, where the mixture may be solidified by removal of the solvent using, e.g., a heat treatment without decomposing the matrix material.

In still further exemplary embodiments of the invention, the use of a medical device as described above as a support for the culturing of cells and/or tissue in vivo or in vitro is provided, for example as a scaffold for tissue engineering, wherein the device may be used a living organism or in a bioreactor.

In other exemplary embodiments of the invention, the composite material which may be used to form at least a portion of a medical device as described above may be produced by a process which can include a solidification procedure. The solidification procedure may include a thermal treatment, drying, freeze-drying, application of a vacuum, e.g. evaporation of the solvent, or cross linking, wherein the cross linking may be induced chemically, thermally or by radiation. The solidification procedure may also include a phase separation in the liquid mixture comprising the reticulating agent and the matrix material into a solid phase and a liquid phase, or precipitation of solids from the liquid mixture, for example, before or during removal of the solvent, and/or by cross linking of the matrix material.

In further exemplary embodiments of the present invention, a phase separation or precipitation which can be used in processes for manufacturing a composite material as described above may be induced by an increase of the viscosity of the liquid mixture comprising the reticulating agent and the matrix material, which may be produced by, for example, cross linking, curing, drying, rapidly increasing the temperature, rapidly lowering the temperature, or rapidly removing the solvent.

In certain exemplary embodiments of the present invention, the matrix material may not decompose substantially during the manufacture of the composite material used to form at least a portion of a medical device.

In still further exemplary embodiments of the present invention, a liquid mixture which may be used in processes for manufacturing a composite material as described above may include at least one cross linker, which may be selected such that cross linking during processing of the liquid mixture before the solidification procedure does not produce a significant viscosity change in the system, and/or the cross linking reaction begins during solidification.

In accordance with exemplary embodiments of the present invention, improved medical devices may be provided which can be formed using a composite material comprising a reticulated, porous structure produced by a process which can provide tailoring and/or adjustment of certain physico-chemical properties of the material, and which may be functionalized for several applications in the field of therapy and diagnosis. The degree of porosity and/or pore sizes of a composite material suitable for coating or forming of medical devices may be selectively adjusted in accordance with exemplary embodiments of the present invention, for example by suitably selecting the amount and type of reticulating agents, their geometry and/or particle size, and/or by combining different particle sizes of the reticulating agent and the matrix material. Adjustment of biocompatibility, thermal coefficient of expansion, the electric, dielectric, conducting or semi-conducting and magnetically or optical properties and/or further physico-chemical properties may be accomplished in accordance with exemplary embodiments of the present invention.

In still further exemplary embodiments of the present invention, a fine structuring of a reticulated composite material with regard to the degree of porosity, the pore size and the morphology may be selectively influenced, e.g., by suitably selecting solidification conditions during manufacture. Additionally, composite materials may be produced for use in medical devices by combining reticulating agents and a suitable matrix material. For example, the mechanical, electrical, thermal and optical properties thereof can be selectively adjusted, e.g., by the solids content of the reticulating agent and/or the matrix material in the liquid mixture, the type of solvent or solvent mixture, the ratio of reticulating agents to matrix material and/or by suitably selecting the materials according to their primary particle size and their structure and type. For example, by suitably selecting conditions in the liquid mixture, e.g., conditions upon solidifying the mixture, the reticulating particles may be oriented in the form of a solid network which can influence and/or determine the porosity and other properties of the resulting composite material.

In exemplary embodiments of the present invention, components and processing conditions used to form a composite material may be selected such that the solids in a liquid mixture may form a self-organizing network structure, e.g., a reticulated structure before and/or during a solidification procedure. Suitable reticulating agents, for example mixtures of reticulating agents of different sizes and/or mixtures of reticulating agent particles with tubes, fibers or wires, may tend to self-aggregate in the liquid mixture, and this aggregation may be enhanced, for example, by suitably selecting the matrix material, the solvent, if any, as well as certain additives, which may produce composite materials especially suited for forming medical devices and/or for providing coatings on such devices.

These and other objects, features and advantages of the present invention will become apparent upon reading the following detailed description of embodiments of the invention, when taken in conjunction with the appended claims.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is an exemplary photographic image at 50,000× magnification of the porous composite material layer described in a first example;

FIG. 2 is an exemplary SEM image at 20,000× magnification of the material described in a second example;

FIG. 3a is an exemplary SEM image at a magnification of 150× of the porous composite material coated stent described in a third example;

FIG. 3b is an exemplary SEM image at a magnification of 1,000× of the porous composite material coated stent described in the third example;

FIG. 3c is an exemplary SEM image at a magnification of 5,000× of the porous composite material coated stent described in the third example;

FIG. 4a is an exemplary SEM image at a magnification of 150× of the porous composite material coated stent described in a fourth example;

FIG. 4b is an exemplary SEM image at a magnification of 1,000× of the porous composite material coated stent described in the fourth example;

FIG. 4c is an exemplary SEM image at a magnification of 20,000× of the porous composite material coated stent described in the fourth example;

FIG. 5a is an exemplary microscopic image of a cell culture grown on the scaffold described in a fifth example at an elapsed time of 120 minutes;

FIG. 5b is an exemplary microscopic image of a cell culture grown on the scaffold described in the fifth example at an elapsed time of 3 days;

FIG. 5c is an exemplary microscopic image of a cell culture grown on the scaffold described in the fifth example at an elapsed time of 5 days;

FIG. 6 is an exemplary image at 100× magnification of the bone replacement material described in a sixth example;

FIG. 7a is an exemplary SEM image at a magnification of 100× of the material described in a seventh example;

FIG. 7b is an exemplary SEM image at a magnification of 20,000× of the material described in the seventh example; and

FIG. 8 provides exemplary images of the material described in an eight example at various magnifications.

### DETAILED DESCRIPTION OF EXEMPLARY EMBODIMENTS

In accordance with certain exemplary embodiments of the present invention, a medical device can be provided which comprises a reticulated porous composite material that may be produced using a process as described herein. The composite material may comprise at least one reticulating agent and at least one matrix material as described herein, and the reticulating agent may be embedded in the matrix material. In exemplary embodiments, at least one, optionally both, of the reticulating agent and the matrix material can be a synthetic material, i.e. a material that is not of natural origin. The composite material may be a rigid, substantially non-elastic material.

The medical device may be provided as an implant, and can be formed using both composite materials in accordance with exemplary embodiments of the present invention, as well as other materials which may be used conventionally to form such implants. Examples of other materials which may be used in conjunction with the composite materials described herein may include, for example, amorphous and/or (partially-) crystalline carbon, solid carbon material, porous carbon, graphite, carbon composite materials, carbon fibers, ceramics such as zeolites, silicates, aluminum oxides, aluminosilicates, silicon carbide, silicon nitride, metal carbides, metal oxides, metal nitrides, metal carbonitrides, metal oxycarbides, metal oxynitrides and metal oxycarbonitrides of the transition metals, such as titanium, zirconium, hafnium, vanadium, niobium, tantalum, chromium, molybdenum, tungsten, manganese, rhenium, iron, cobalt, nickel; metals and metal alloys, in particular the noble metals such as gold, silver, ruthenium, rhodium, palladium, osmium, iridium, platinum; metals and metal alloys of titanium, zirconium, hafnium, vanadium, niobium, tantalum, chromium, molybdenum, tungsten, manganese, rhenium, iron, cobalt, nickel, copper; steel, in particular stainless steel, memory alloys such as nitinol, nickel titanium alloy, glass, stone, glass fibers, minerals, natural or synthetic bone substance, imitation bone based on alkaline earth metal carbonates such as calcium carbonate, magnesium carbonate, strontium carbonate, apatite minerals such as hydroxyl apatite, foamed materials such as polymer foams, foamed ceramics and the like, materials which may be dissolvable under physiologic conditions such as, e.g., magnesium, zinc or alloys comprising magnesium and/or zinc, as well as any combinations of the aforementioned materials and/or combinations thereof with the porous composite material as described herein.

In an exemplary embodiment of the present invention, a medical device may be provided in the form of stent which comprises a material capable of being dissolved under physiologic conditions such as, for example, magnesium, zinc or an alloy comprising magnesium and/or zinc. The device may further include a composite material, which may be in the form of a coating, and which can be radiopaque, or which may include a marker, for example, a metal or metal particles such as silver or gold. The coating may be rapidly dissolved or peeled off from the device, for example a stent, after implantation under physiologic conditions, allowing a transient marking to occur. The composite material may further be loaded with therapeutically active ingredients.

The processes for manufacturing the composite material of the medical devices described herein can lead to the formation of a reticulated porous structure, which may have an influence on certain macroscopic properties of the composite material and of a device which includes such a material. Therefore, properties of medical devices in accordance with exemplary embodiments of the present invention, and of composite materials which may be used to form them, may be better understood by referring to the techniques and materials which may be used for their manufacture.

In an exemplary process for manufacturing a medical device in accordance with certain exemplary embodiments of the present invention, a mixture capable of flowing can be prepared comprising at least one reticulating agent and at least one matrix material which may be a polymer or a combination of polymers that can be subsequently solidified. Solidification may occur, for example, by curing, cross linking, hardening or drying without significant decomposition of the matrix material, which may essentially retain its structural integrity. The mixture may include a liquid composition provided in the form of a dispersion, suspension, emulsion or solution, optionally comprising a solvent or solvent mixture.

In an exemplary embodiment of the present invention, the mixture may be substantially free of any solvents and may utilize a liquid matrix material, which can be a material in a molten state, e.g., a melt of the matrix material.

The terms “liquid mixture” and “mixture capable of flowing” can be understood to refer to a mixture capable of flowing, which may contain or may exclude a solvent, and which may include melts, slurries or pasty materials having a high viscosity, or substantially dry flowable powder or particle mixtures.

A liquid mixture may be prepared using conventional techniques, e.g., by dissolving or dispersing one or more solid components in a solvent or a matrix material in any suitable order, by mixing solids in a dry state and optionally adding one or more solvents, by melting a matrix material and dispersing one or more reticulating agents therein, optionally before adding a solvent, or by preparing a paste or slurry and subsequently diluting it with a solvent or a dispersion of other components in a solvent.

The term “reticulating agent” can refer to a material that can be oriented into a network or network-like structure under certain conditions described herein for converting a liquid mixture into a porous solidified composite material. In exemplary embodiments of the present invention, reticulating agents can include materials that are capable of self-orienting or promoting self-orientation into a network or network-like structure. A “network” or “network-like structure” can be understood to refer to a regular and/or irregular three-dimensional arrangement which may have void space or pores within it. The porous structure of a composite material as described herein may, e.g., permit or promote growth of biological tissue thereon and/or proliferation of the tissue into the material. The porous structure may also be used, for example, to store and/or release active agents, diagnostic markers and the like.

A reticulating agent may comprise organic and/or inorganic materials of any suitable form or size, or any mixtures thereof. For example, the reticulating agent(s) may include inorganic materials such as, for example, zero-valent metals, metal powders, metal compounds, metal alloys, metal oxides, metal carbides, metal nitrides, metal oxynitrides, metal carbonitrides, metal oxycarbides, metal oxynitrides, metal oxycarbonitrides, organic or inorganic metal salts, including salts from alkaline and/or alkaline earth metals and/or transition metals, including alkaline or alkaline earth metal carbonates, -sulfates, -sulfites, semi conductive metal compounds, including those of transition metals and/or metals from the main group of the periodic system; metal based core-shell nanoparticles, glass or glass fibers, carbon or carbon fibers, silicon, silicon oxides, zeolites, titanium oxides, zirconium oxides, aluminum oxides, aluminum silicates, talcum, graphite, soot, flame soot, furnace soot, gaseous soot, carbon black, lamp black, minerals, phyllosilicates, or any mixtures thereof.

Reticulating agents may also be biodegradable metal-based compositions, for example, alkaline or alkaline earth metal salts or compounds such as magnesium-based or zinc-based compounds or the like, or nano-alloys or any mixture thereof. The reticulating agents used in certain exemplary embodiments of the present invention may include magnesium salts, oxides or alloys, which can be used in biodegradable coatings or molded bodies, and which can be provided in the form of an implant or a coating on an implant that may be capable of degradation when exposed to bodily fluids, and which may further result in formation of magnesium ions and hydroxylapatite.

Reticulating agents may also include, but are not limited to, powders, including nanomorphous nanoparticles; of zero-valent-metals, metal oxides or combinations thereof, e.g., metals and metal compounds which may be selected from the main group of metals in the periodic table, transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or rare earth metals. Metal-based compounds which may be used include, e.g., organometallic compounds, metal alkoxides, carbon particles such as, for example soot, lamp-black, flame soot, furnace soot, gaseous soot, carbon black, graphite, carbon fibers or diamond particles, and the like. Reticulating agents may also include metal-containing endohedral fullerenes and/or endometallofullerenes, including those comprising rare earth metals such as, e.g., cerium, neodymium, samarium, europium, gadolinium, terbium, dysprosium, holmium, iron, cobalt, nickel, manganese or mixtures thereof such as iron-platinum-mixtures or alloys. Magnetic super paramagnetic or ferromagnetic metal oxides may also be used including, e.g., iron oxides or ferrites, e.g. cobalt-, nickel- or manganese ferrites. Magnetic metals or alloys may be used to provide materials having magnetic, super-paramagnetic, ferromagnetic or signaling properties, such as ferrites, e.g. gamma-iron oxide, magnetite or ferrites of Co, Ni, or Mn. Examples of such materials are described in International Patent Publications WO83/03920, WO83/01738, WO88/00060, WO85/02772, WO89/03675, WO90/01295 and WO90/01899, and U.S. Pat. Nos. 4,452,773, 4,675,173 and 4,770,183. A reticulating agent can include any combination of materials listed herein.

Additionally, semiconducting compounds and/or nanoparticles may be used as a reticulating agent in further exemplary embodiments of the present invention, including semiconductors of groups II-VI, groups III-V, or group IV of the periodic system. Suitable group I-VI-semiconductors include, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples of group III-V semiconductors include, for example, GaAs, GaN, GaP, GaSb, InGaAs, InP, InN, InSb, InAs, AlAs, AlP, AlSb, AlS, or mixtures thereof. Examples of group IV semiconductors include germanium, lead and silicon. Also, combinations of any of the foregoing semiconductors may be used.

In certain exemplary embodiments of the present invention, it may be preferable to use complex metal-based nanoparticles as the reticulating agents. These may include, for example, so-called core/shell configurations such as those described by Peng et al., Epitaxial Growth of Highly Luminescent CdSe/CdS Core/Shell Nanoparticles with Photostability and Electronic Accessibility, Journal of the American Chemical Society (1997, 119: 7019-7029). The core of semiconducting nanoparticles having a core-shell configuration may have a diameter of about 1 to 30 nm, or preferably about 1 to 15 nm, upon which further semiconducting nanoparticles may be crystallized to a depth of about 1 to 50 monolayers, or preferably about 1 to 15 monolayers. Cores and shells may be present in combinations of the materials listed above, including CdSe or CdTe cores, and CdS or ZnS shells.

In a further exemplary embodiment of the present invention, the reticulating agents may be selected based on their absorptive properties for radiation in a wavelength ranging anywhere from gamma radiation up to microwave radiation, or based on their ability to emit radiation, particularly in the wavelength region of about 60 nm or less. By suitable selection of a reticulating agent, materials having non-linear optical properties may be produced. These include, for example, materials that can block IR-radiation of specific wavelengths, which may be suitable for marking purposes or to form therapeutic radiation-absorbing implants. The reticulating agents, their particle sizes and the diameter of their core and shell may be selected to provide photon emitting compounds, such that the emission is in the range of about 20 nm to 1000 nm. Alternatively, a mixture of suitable compounds may be selected which is capable of emitting photons of differing wavelengths when exposed to radiation. In one exemplary embodiment of the present invention, fluorescent metal-based compounds may be selected that do not require quenching.

In exemplary embodiments of the present invention, the reticulating agent may include carbon species such as nanomorphous carbon species, for example, fullerenes such as C36, C60, C70, C76, C80, C86, C112, etc., or any mixtures thereof. Reticulating agents may also include multi-, double- or single-walled nanotubes, such as MWNT, DWNT or SWNT, randomly-oriented nanotubes, as well as so-called fullerene onions or metallo-fullerenes, or graphite, soot, carbon black and the like.

Additionally, materials that may be used as reticulating agents to produce medical devices in accordance with exemplary embodiments of the present invention may include organic materials such as polymers, oligomers or pre-polymers; shellac, cotton, or fabrics; or any combinations thereof.

In certain exemplary embodiments of the present invention, the reticulating agent may comprise a mixture of at least one inorganic material and at least one organic material.

Reticulating agents comprising one or more of the materials mentioned herein may be provided in the form of particles which may have an essentially spherical or sphere-like irregular shape, or fibers. They may also be provided in the form of nano- or microcrystalline particles, powders or nanowires. The reticulating agents may have an average particle size of about 1 nm to about 1,000 μm, preferably about 1 nm to 300 μm, or more preferably from about 1 nm to 6 μm.

The reticulating agents may comprise at least two particles which may be formed of the same or different material, and which may have sizes that differ by a factor of greater than about 2, or greater than about 3, 5, or 10. Differences in particle sizes of reticulating agents may promote self-orientation of the agents when forming a network structure.

In exemplary embodiments of the present invention, the reticulating agents may include a combination of carbon particles such as soot, carbon black or lamp black, with fullerenes or fullerene mixtures. The carbon particles may have an average size ranging from about 50 to 200 nm, or from about 90 to 120 nm. The reticulating agent may alternatively or additionally include a combination of metal oxide particles such as, for example, silica, alumina, titanium oxide, zirconium oxide, or zeolites or combinations thereof, with fullerenes or fullerene mixtures. The metal oxide particles may have an average size ranging from about 5 to 150 nm, or from about 10 to 100 nm. In certain exemplary embodiments of the present invention, the reticulating agent may include a combination of one or more metal powders with metal oxide particles such as, e.g., silica, alumina, titanium oxide, zirconium oxide, zeolites or combinations thereof. The metal oxide particles may have an average size ranging from about 5 to 150 nm, or from about 10 to 100 nm, and the metal powder may have an average particle size in the micrometer range, e.g. from about 0.5 to 10 μm, or from about 1 to 5 μm. Reticulating agents can be combined with a matrix material such as, e.g., epoxy resins, which may be thermally curable and/or cross linkable phenoxy resins.

Alternatively, or in addition, the reticulating agent can be provided in the form of tubes, fibers, fibrous materials or wires, including nanowires, which can comprise any of the materials mentioned above. Examples of such agents may include carbon fibers, nanotubes, glass fibers, metal nanowires or metal microwires. These reticulating agents can have an average length of about 5 nm to 1,000 μm, or about 5 nm to 300 μm, or preferably about 5 nm to 10 μm, or about 2 to 20 μm, and/or an average diameter between about 1 nm and 1 μm, about 1 nm to 500 nm, preferably about 5 nm to 300 nm, or about 10 to 200 nm.

The particle sizes can be provided as a mean or average particle size, which may be determined by laser techniques such as a TOT-method (Time-Of-Transition), which may be determined, e.g., on a CIS Particle Analyzer of Ankersmid. Further suitable techniques for determining particle size can include powder diffraction techniques or TEM (Transmission Electron Microscopy) techniques.

In certain exemplary embodiments of the present invention, solvent-free mixtures may be used, wherein the matrix material may include, for example, a liquid prepolymer or a melt, e.g., a molten matrix material, which can be subsequently solidified by techniques such as cross linking or curing.

In further exemplary embodiments of the present invention, the reticulating agent and the matrix material may exclude fibers or fibrous materials, and the resulting composite used in the medical device may be substantially free of fibers.

In further exemplary embodiments of the present invention, the reticulating agents may be modified, e.g., to improve their dispersibility or wettability in solvents or in the matrix material, to generate additional functional properties or to increase compatibility between the agent and the matrix. Conventional techniques may be used to modify the particles or fibers, if necessary, depending on the requirements of the particular composition and the materials used. For example, silane compounds such as organosilanes may be used to modify the reticulating agents. Examples of suitable organosilanes and other modifying agents are described, for example, in International Patent Application No. PCT/EP2006/050622 and U.S. patent application Ser. No. 11/346,983, filed Feb. 3, 2006, and these may be employed in exemplary embodiments of the present invention, as well as compositions described in such applications and herein as cross linkers.

In certain exemplary embodiments of the present invention, the reticulating agents may be modified with alkoxides, metal alkoxides, colloidal particles and/or metal oxides and the like. The metal alkoxides may have the general formula M(OR)ₓ where M is any metal from a metal alkoxide that may, e.g., hydrolyze and/or polymerize in the presence of water. R may be an alkyl radical comprising between 1 and about 30 carbon atoms, which may be straight, chained or branched, and x can have a value equivalent to the metal ion valence. Metal alkoxides such as Si(OR)₄, Ti(OR)₄, Al(OR)₃, Zr(OR)₃ and Sn(OR)₄ may also be used. R may also be a methyl, ethyl, propyl or butyl radical. Further examples of suitable metal alkoxides can include Ti(isopropoxy)₄, Al(isopropoxy)₃, Al(sec-butoxy)₃, Zr(n-butoxy)₄ and Zr(n-propoxy)₄.

Further suitable modifying agents can include, e.g., silicon alkoxides such as tetraalkoxysilanes, wherein the alkoxy may be branched or straight-chained and may contain about 1 to 25 carbon atoms, e.g. tetramethoxysilane (TMOS), tetraethoxysilane (TEOS) or tetra-n-propoxysilane, as well as oligomeric forms thereof. Other suitable agents can include alkylalkoxysilanes, wherein the alkoxy may again be branched or straight-chained and may contain about 1 to 25 carbon atoms, and alkyl may be a substituted or unsubstituted, branched or straight-chain alkyl having about 1 to 25 carbon atoms, e.g., methyltrimethoxysilane (MTMOS), methyltriethoxysilane, ethyl-triethoxysilane, ethyltrimethoxysilane, methyltripropoxysilane, methyltributoxysilane, propyltrimethoxysilane, propyltriethoxysilane, isobutyltriethoxysilane, isobutyl-trimethoxysilane, octyltriethoxysilane, octyltrimethoxysilane (commercially available from Degussa AG, Germany), methacryloxydecyltrimethoxysilane (MDTMS); aryltrialkoxysilanes such as phenyltrimethoxysilane (PTMOS), phenyltriethoxysilane, which is commercially available from Degussa AG, Germany; phenyltripropoxysilane, and phenyltributoxysilane, phenyl-tri-(3-glycidyloxy)-silane-oxide (TGPSO), 3-aminopropyltrimethoxysilane, 3-aminopropyl-triethoxysilane, 2-aminoethyl-3-aminopropyltrimethoxysilane, triaminofunctional propyltrimethoxysilane (Dynasylan® TRIAMO, available from Degussa AG, Germany), N-(n-butyl)-3-aminopropyltrimethoxysilane, 3-aminopropylmethyl-diethoxysilane, 3-glycidyl-oxypropyltrimethoxysilane, 3-glycidyloxypropyltriethoxy-silane, vinyltrimethoxysilane, vinyltriethoxysilane, 3-mercaptopropyltrimethoxy-silane, Bisphenol-A-glycidylsilanes; (meth)acrylsilanes, phenylsilanes, oligomeric or polymeric silanes, epoxysilanes; fluoroalkylsilanes such as fluoroalkyltrimethoxysilanes, fluoroalkyltriethoxysilanes which may have a partially or fully fluorinated, straight-chain or branched fluoroalkyl residue containing about 1 to 20 carbon atoms, e.g., tridecafluoro-1,1,2,2-tetrahydrooctyltriethoxysilane, or modified reactive fluoroalkylsiloxanes (available from Degussa AG, Germany, under the trademarks Dynasylan® F8800 and F8815); or any mixtures thereof. Compositions such as 6-amino-1-hexanol, 2-(2-aminoethoxy)ethanol, cyclohexyl-amine, butyric acid cholesterylester (PCBCR), 1-(3-methoxycarbonyl)-propyl)-1-phenylester, or combinations thereof, may also be used as modifyin agents.

The modification agents and silanes listed above may optionally also be used as cross linkers, e.g., in a solidification procedure for curing and/or hardening of the liquid mixture.

In a further exemplary embodiment of the present invention, the reticulating agent can include particles or fibers which may comprise polymers, oligomers and/or pre-polymeric organic materials. These particles or fibers may be prepared by conventional polymerization techniques capable of producing discrete particles, e.g. polymerizations in liquid media in emulsions, dispersions, suspensions or solutions. Furthermore, these particles or fibers may also be produced by extrusion, spinning, pelletizing, milling, or grinding of polymeric materials. A reticulating agent comprising particles or fibers of polymers, oligomers, pre-polymers, thermoplastics or elastomers may also be used with homopolymers or copolymers of these components for use as a matrix material. These polymers may be used as the matrix material if not in the form of a particle or a fiber, or as a reticulating agent if they are provided in the form of a particle or a fiber. Polymeric reticulating agents may be capable of decomposing at elevated temperatures, and may thus act as pore formers in the composite materials produced. Examples of such exemplary agents may include, e.g., polyolefins such as polyethylene or polypropylene in the form of particles and/or fibers.

In exemplary embodiments of the present invention, the reticulating agent may include electrically conducting polymers, such as those described herein below as electrically conductive matrix materials.

In further exemplary embodiments of the present invention, the reticulating agent may include, e.g., polymer-encapsulated non-polymeric particles, wherein the non-polymeric particles may include the materials listed above. Techniques and polymerization reactions for encapsulating the non-polymeric reticulating agent particles can include any suitable conventional polymerization reaction, for example, a radical or non-radical polymerization, enzymatic or non-enzymatic polymerization, or a poly-condensation reaction. The encapsulation of reticulating agent particles can, depending from the individual components used, lead to covalently or non-covalently encapsulated reticulating agent particles. Encapsulated reticulating agents may be provided in the form of polymer spheres, particularly nanosized or micro spheres, or in the form of dispersed, suspended or emulgated particles or capsules, respectively, for combination with the matrix material. Conventional techniques can be utilized to form the polymer-encapsulated particles. Suitable encapsulation techniques and materials and conditions used therewith are described, for example, in International Patent Application Nos. PCT/EP2006/060783 and PCT/EP2006/050373, and in U.S. patent application Ser. No. 11/385,145 filed Mar. 20, 2006, and Ser. No. 11/339,161, filed Jan. 24, 2006.

Suitable encapsulation methods are described, for example, in Australian Patent Application No. AU 9169501, European Patent Publication Nos. EP 1205492, EP 1401878, EP 1352915 and EP 1240215, U.S. Pat. No. 6,380,281, U.S. Patent Publication No. 2004/192838, Canadian Patent Publication No. CA 1336218, Chinese Patent Publication No. CN 1262692T, British Patent Publication No. GB 949722, German Patent Publication No. DE 10037656, and in International Patent Application Nos. PCT/EP2006/060783 and PCT/EP2006/050373 as mentioned above.

The encapsulated reticulating agents may be produced in a size of about 1 nm to 500 nm, or in the form of microparticles having an average size between about 5 nm to 5 μm. Reticulating agents may be further encapsulated in mini- or micro-emulsions of suitable polymers. The term “mini-emulsion” or “micro-emulsion” may be understood to refer to a dispersion comprising an aqueous phase, an oil or hydrophobic phase, and one or more surface-active substances. Such emulsions may comprise suitable oils, water, one or several surfactants, optionally one or several co-surfactants and/or one or several hydrophobic substances. Mini-emulsions may comprise aqueous emulsions of monomers, oligomers or other pre-polymeric reactants stabilized by surfactants, which may be easily polymerized, and wherein the particle size of the emulgated droplets can be between about 10 nm and 500 nm or larger.

Mini-emulsions of encapsulated reticulating agents can also be made from non-aqueous media, for example, formamide, glycol or non-polar solvents. Pre-polymeric reactants may comprise thermosets, thermoplastics, plastics, synthetic rubbers, extrudable polymers, injection molding polymers, moldable polymers, and the like, or mixtures thereof, including pre-polymeric reactants from which poly(meth)acrylics can be used.

Examples of suitable polymers for encapsulating the reticulating agents can include, but are not limited to, homopolymers or copolymers of aliphatic or aromatic polyolefins such as polyethylene, polypropylene, polybutene, polyisobutene, polypentene; polybutadiene; polyvinyls such as polyvinyl chloride or polyvinyl alcohol, poly(meth)acrylic acid, polymethylmethacrylate (PMMA), polyacrylocyano acrylate; polyacrylonitril, polyamide, polyester, polyurethane, polystyrene, polytetrafluoroethylene; particularly preferred may be biopolymers such as collagen, albumin, gelatin, hyaluronic acid, starch, celluloses such as methylcellulose, hydroxypropyl cellulose, hydroxypropyl methylcellulose, carboxymethylcellulose phthalate; casein, dextranes, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate), shellac, and combinations of these homopolymers or copolymers; but may exclude cyclodextrine and derivatives thereof or similar carrier systems.

Other encapsulating materials that may be used include poly(meth)acrylate, unsaturated polyester, saturated polyester, polyolefines such as polyethylene, polypropylene, polybutylene, alkyd resins, epoxypolymers, epoxy resins, polyamide, polyimide, polyetherimide, polyamideimide, polyesterimide, polyesteramideimide, polyurethane, polycarbonate, polystyrene, polyphenol, polyvinylester, polysilicone, polyacetale, cellulosic acetate, polyvinylchloride, polyvinylacetate, polyvinylalcohol, polysulfone, polyphenylsulfone, polyethersulfone, polyketone, polyetherketone, polybenzimidazole, polybenzoxazole, polybenzthiazole, polyfluorocarbons, polyphenylenether, polyarylate, cyanatoester-polymere, or mixtures or copolymers of any of the foregoing.

In certain exemplary embodiments of the present invention, the polymers used to encapsulate the reticulating agents may comprise mono(meth)acrylate-, di(meth)acrylate-, tri(meth)acrylate-, tetra-acrylate- and pentaacrylate-based poly(meth)acrylates. Examples for suitable mono(meth)acrylates are hydroxyethyl acrylate, hydroxyethyl methacrylate, hydroxypropyl methacrylate, hydroxypropyl acrylate, 3-chloro-2-hydroxypropyl acrylate, 3-chloro-2-hydroxypropyl methacrylate, 2,2-dimethylhydroxypropyl acrylate, 5-hydroxypentyl acrylate, diethylene glycol monoacrylate, trimethylolpropane monoacrylate, pentaerythritol monoacrylate, 2,2-dimethyl-3-hydroxypropyl acrylate, 5-hydroxypentyl methacrylate, diethylene glycol monomethacrylate, trimethylolpropane monomethacrylate, pentaerythritol monomethacrylate, hydroxy-methylated N-(1,1-dimethyl-3-oxobutyl)acrylamide, N-methylolacrylamide, N-methylolmethacrylamide, N-ethyl-N-methylolmethacrylamide, N-ethyl-N-methylolacrylamide, N,N-dimethylol-acrylamide, N-ethanolacrylamide, N-propanolacrylamide, N-methylolacrylamide, glycidyl acrylate, and glycidyl methacrylate, methyl acrylate, ethyl acrylate, propyl acrylate, butyl acrylate, amyl acrylate, ethylhexyl acrylate, octyl acrylate, t-octyl acrylate, 2-methoxyethyl acrylate, 2-butoxyethyl acrylate, 2-phenoxyethyl acrylate, chloroethyl acrylate, cyanoethyl acrylate, dimethylaminoethyl acrylate, benzyl acrylate, methoxybenzyl acrylate, furfuryl acrylate, tetrahydrofurfuryl acrylate and phenyl acrylate; di(meth)acrylates may be selected from 2,2-bis(4-methacryloxyphenyl)propane, 1,2-butanediol-diacrylate, 1,4-butanediol-diacrylate, 1,4-butanediol-dimethacrylate, 1,4-cyclohexanediol-dimethacrylate, 1,10-decanediol-dimethacrylate, diethylene-glycol-diacrylate, dipropyleneglycol-diacrylate, dimethylpropanediol-dimethacrylate, triethyleneglycol-dimethacrylate, tetraethyleneglycol-dimethacrylate, 1,6-hexanediol-diacrylate, Neopentylglycol-diacrylate, polyethyleneglycol-dimethacrylate, tripropyleneglycol-diacrylate, 2,2-bis[4-(2-acryloxyethoxy)phenyl]propane, 2,2-bis[4-(2-hydroxy-3-methacryloxypropoxy)phenyl]-propane, bis(2-methacryloxyethyl)N,N-1,9-nonylene-biscarbamate, 1,4-cycloheane-dimethanol-dimethacrylate, and diacrylic urethane oligomers; tri(meth)acrylates may be selected from tris(2-hydroxyethyl)isocyanurate-trimethacrylate, tris(2-hydroxyethyl)isocyanurate-triacrylate, trimethylolpropane-trimethacrylate, trimethylol-triacrylate or pentaerythritol-triacrylate; tetra(meth)acrylates may be selected from pentaerythritol-tetraacrylate, di-trimethylopropane-tetraacrylate, or ethoxylated pentaerythritol-tetraacrylate; suitable penta(meth)acrylates may be selected from dipentaerythritol-pentaacrylate or pentaacrylate-esters; as well as mixtures, copolymers or combinations of any of the foregoing. Biopolymers or acrylics may be used to encapsulate the reticulating agents in certain exemplary embodiments of the present invention, e.g., for biological or medical applications.

Encapsulating polymer reactants may comprise polymerizable monomers, oligomers or elastomers such as polybutadiene, polyisobutylene, polyisoprene, poly(styrene-butadiene-styrene), polyurethanes, polychloroprene, natural rubber materials, gums such as gum arabica, locust bean gum, gum caraya, or silicone, and mixtures, copolymers or any combinations thereof. The reticulating agents may be encapsulated in elastomeric polymers alone, or in mixtures of thermoplastic and elastomeric polymers, or in an alternating sequence of thermoplastic and elastomeric shells or layers.

The polymerization reaction for encapsulating the reticulating agents can include any suitable conventional polymerization reaction, for example, a radical or non-radical polymerization, enzymatic or non-enzymatic polymerization, including poly-condensation reactions. The emulsions, dispersions or suspensions used may be in the form of aqueous, non-aqueous, polar or homopolar systems. By adding suitable surfactants, the amount and size of the emulgated or dispersed droplets can be adjusted as required.

The surfactants may be anionic, cationic, zwitter-ionic or non-ionic surfactants or any combinations thereof. Preferred anionic surfactants may include, but are not limited to, soaps, alkylbenzolsulfonates, alkansulfonates, olefinsulfonates, alkyethersulfonates, glycerinethersulfonates, α-methylestersulfonates, sulfonated fatty acids, alkylsulfates, fatty alcohol ether sulfates, glycerine ether sulfates, fatty acid ether sulfates, hydroxyl mixed ether sulfates, monoglyceride(ether)sulfates, fatty acid amide(ether)sulfates, mono- and di-alkylsulfosuccinates, mono- and dialkylsulfosuccinamates, sulfotriglycerides, amidsoaps, ethercarboxylicacid and their salts, fatty acid isothionates, fatty acid arcosinates, fatty acid taurides, N-acylaminoacid such as acyllactylates, acyltartrates, acylglutamates and acylaspartates, alkyloligoglucosidsulfates, protein fatty acid condensates, including plant derived products based on wheat; and alky(ether)phosphates.

Cationic surfactants suitable for encapsulation reactions in certain embodiments of the present invention may comprise quaternary ammonium compounds such as, for example, dimethyldistearylammoniumchloride, Stepantex® VL 90 (Stepan), esterquats, particularly quaternized fatty acid trialkanolaminester salts, salts of long-chain primary amines, quaternary ammonium compounds such as hexadecyltrimethyl-ammoniumchloride (CTMA-Cl), Dehyquart® A (cetrimoniumchloride, Cognis), or Dehyquart® LDB 50 (lauryldimethylbenzyl ammonium chloride, Cognis).

Other preferred surfactants may include lecithin, poloxamers, e.g., block copolymers of ethylene oxide and propylene oxide, including those available from BASF Co. under the trade name pluronic®, including pluronic® F68NF, alcohol ethoxylate based surfactants from the TWEEN® series available from Sigma Aldrich or Krackeler Scientific Inc., and the like.

The reticulating agent can be added before or during the start of the polymerization reaction and may be provided in the form of a dispersion, emulsion, suspension or solid solution, or as solution of the reticulating agents in a suitable solvent or solvent mixture, or any mixtures thereof. The encapsulation process may comprise the polymerization reaction, optionally with the use of initiators, starters or catalysts, where an in-situ encapsulation of the reticulating agents in polymer capsules, spheroids or droplets may occur. The solids content of the reticulating agents in such encapsulation mixtures may be selected such that the solids content in the polymer capsules, spheroids or droplets is between about 10 weight % and about 80 weight % of active agent within the polymer particles.

Optionally, the reticulating agents may also be added after completion of the polymerization reaction, either in solid form or in liquid form. The reticulating agents can be selected from those compounds that are able to bind to the polymer spheroids or droplets, either covalently or non-covalently. The droplet size of the polymers and the solids content of reticulating agents can be selected such that the solids content of the reticulating agent particles ranges from about 5 weight % to about 90 weight % with respect to the total weight of the polymerization mixture.

In an exemplary embodiment of the present invention, the encapsulation of the reticulating agents during the polymerization can be repeated at least once by addition of further monomers, oligomers or pre-polymeric agents after completion of a first polymerization/encapsulation step. By performing at least one repeated polymerization step in this manner, multilayer coated polymer capsules can be produced. Also, reticulating agents bound to polymer spheroids or droplets may be encapsulated by subsequently adding monomers, oligomers or pre-polymeric reactants to overcoat the reticulating agents with a polymer capsule. Repetition of such processes can produce multilayered polymer capsules comprising the reticulating agent.

Any of the encapsulation steps described above may be combined with each other. In a preferred exemplary embodiment of the present invention, polymer-encapsulated reticulating agents can be further coated with release-modifying agents.

In further exemplary embodiments of the present invention, the reticulating agents or polymer encapsulated reticulating agents may be further encapsulated in vesicles, liposomes or micelles, or overcoatings. Suitable surfactants for this purpose may include the surfactants typically used in encapsulation reactions as described in above. Further Surfactants include compounds having hydrophobic groups which may include hydrocarbon residues or silicon residues, for example, polysiloxane chains, hydrocarbon based monomers, oligomers and polymers, or lipids or phospholipids, or any combinations thereof, particularly glycerylester such as phosphatidylethanolamine, phosphatidylcholine, polyglycolide, polylactide, polymethacrylate, polyvinylbuthylether, polystyrene, polycyclopentadienyl-methylnorbornene, polypropylene, polyethylene, polyisobutylene, polysiloxane, or any other type of surfactant.

Furthermore, depending on the polymeric shell which may be provided, surfactants for encapsulating the polymer encapsulated reticulating agents in vesicles, overcoats and the like may be selected from hydrophilic surfactants or surfactants having hydrophilic residues or hydrophilic polymers such as polystyrensulfonicacid, poly-N-alkylvinylpyridinium-halogenide, poly(meth)acrylic acid, polyaminoacids, poly-N-vinylpyrrolidone, polyhydroxyethylmethacrylate, polyvinylether, polyethylenglycol, polypropylene oxide, polysaccharides such as agarose, dextrane, starch, cellulose, amylase, amylopektine or polyethylenglycole, or polyethylennimine of a suitable molecular weight. Also, mixtures from hydrophobic or hydrophilic polymer materials or lipid polymer compounds may be used for encapsulating the polymer capsulated reticulating agents in vesicles or for further over-coating the polymer encapsulating reticulating agents.

Additionally, the encapsulated reticulating agents may be chemically modified by functionalization with suitable linker groups or coatings. For example, they may be functionalized with organosilane compounds or organo-functional silanes. Such compounds which may be suitable for modification of the polymer encapsulated reticulating agents are further described herein above.

The incorporation of polymer-encapsulated particles into the materials described herein can be regarded as a particular form of a reticulation agent. The particle size and particle size distribution of the polymer-encapsulated reticulating agent particles in dispersed or suspended form may correspond to the particle size and particle size distribution of the particles of finished polymer-encapsulated particles. The particle size and monodispersity of polymer-encapsulated particles can be characterized, e.g., by dynamic light scattering techniques applied to the particles suspended in a liquid phase.

Furthermore, the particles used as the reticulating agents in processes in accordance with exemplary embodiments of the present invention may be encapsulated in biocompatible polymers, which may include biodegradable polymers. For example, the biocompatible polymers mentioned herein as possible matrix materials may be used to encapsulate particles. These materials may also be directly used as reticulating agents, as discussed herein above.

Polymers that are pH-sensitive may be used for encapsulating reticulating agent particles or to form reticulating agent particles. For example, the pH-sensitive polymers mentioned herein as possible matrix materials may be used. Furthermore, polysaccharides such as cellulose acetate-phtalate, hydroxypropylmethylcellulose-phtalate, hydroxypropyl-methylcellulose-succinate, cellulose acetate-trimellitate and chitosan may be used.

Temperature-sensitive polymers and/or polymers having a thermogel characteristic may also be used for encapsulating the reticulating agent particles or as the reticulating agent particle itself. Examples of such polymers are described herein below as suitable components of matrix materials.

A reticulating agent such as, for example, a plurality of polymer encapsulated particles or polymer particles used as a reticulating agent, may be combined with a matrix material in a suitable solvent before subsequently being converted into a porous reticulated composite material in accordance with exemplary embodiments of the present invention.

A reticulating agent may be combined with matrix materials, for example, it may be embedded in a matrix material, to form a composite material that can be used to form a medical device. The composite material may be produced in the presence or absence of a suitable solvent or solvent mixture, wherein the matrix materials may be combined with the selected reticulating agents or mixtures thereof to form the porous reticulated composite material.

The matrix material may include polymers, oligomers, monomers or pre-polymerized forms, optionally of synthetic origin, and the polymers may include the polymeric materials mentioned herein above as being suitable for reticulating agents and/or those described in the documents referenced herein as being suitable for encapsulating the reticulating agents, as well as other substances which may be synthesized to form pre-polymeric, partially polymerized or polymeric materials or which are already present as such materials, including polymer composites. Polymer composites may be provided as nano-composites or may contain nanomorphous particles in homogeneously dispersed form, as well as in the form of substances which can be solidified from suspensions, dispersions or emulsions and which may be suitable for forming a composite material with the selected reticulating agents. The polymers used may include thermosets, thermoplastics, synthetic rubbers, extrudable polymers, injection molding polymers, moldable polymers and the like, or mixtures thereof.

Additives may also be utilized which can improve the compatibility of the components used in producing the composite material, for example coupling agents such as silanes, surfactants or fillers, which may be organic or inorganic.

In an exemplary embodiment of the present invention, the polymer provided to form the matrix material may include homopolymers, copolymers, prepolymeric forms and/or oligomers of aliphatic or aromatic polyolefins such as polyethylene, polypropylene, polybutene, polyisobutene, polypentene; polybutadiene; polyvinyls such as polyvinyl chloride, polyvinylacetate, or polyvinyl alcohol, polyacrylates such as poly(meth)acrylic acid, polymethylmethacrylate (PMMA), polyacrylocyano acrylate; polyacrylonitril, polyamide, polyester, polyurethane, polystyrene, polytetrafluoroethylene; bio-compatible polymers as described herein; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate), or shellac, and/or combinations of the above.

In further exemplary embodiments of the present invention, the polymer provided to form the matrix material may include unsaturated or saturated polyesters, alkyd resins, epoxy-polymers, epoxy resins, phenoxy resins, nylon, polyimide, polyetherimide, polyamideimide, polyesterimide, polyesteramideimide, polyurethane, polycarbonate, polystyrene, polyphenol, polyvinylester, polysilicon, polyacetal, cellulose acetate, polysulfone, polyphenylsulfone, polyethersulfone, polyketone, polyetherketone, polyetheretherketone, polyetherketonketones, polybenzimidazole, polybenzoxazole, polybenzthiazole, polyfluorocarbons, polyphenylenether, polyarylate, cyanatoester-polymers, and/or copolymers or mixtures of any of these.

Other suitable polymers that may be used to form the matrix material include acrylics, e.g., mono(meth)acrylate-, di(meth)acrylate-, tri(meth)acrylate-, tetra-acrylate and pentaacrylate-based poly(meth)acrylates. Examples of suitable mono(meth)acrylates may include hydroxyethyl acrylate, hydroxyethyl methacrylate, hydroxypropyl methacrylate, hydroxypropyl acrylate, 3-chloro-2-hydroxypropyl acrylate, 3-chloro-2-hydroxypropyl methacrylate, 2,2-dimethylhydroxypropyl acrylate, 5-hydroxypentyl acrylate, diethylene glycol monoacrylate, trimethylolpropane monoacrylate, pentaerythritol monoacrylate, 2,2-dimethyl-3-hydroxypropyl acrylate, 5-hydroxypentyl methacrylate, diethylene glycol monomethacrylate, trimethylolpropane monomethacrylate, pentaerythritol monomethacrylate, hydroxy-methylated N-(1,1-dimethyl-3-oxobutyl)acrylamide, N-methylolacrylamide, N-methylolmethacrylamide, N-ethyl-N-methylolmethacrylamide, N-ethyl-N-methylolacrylamide, N,N-dimethylol-acrylamide, N-ethanolacrylamide, N-propanolacrylamide, N-methylolacrylamide, glycidyl acrylate, and glycidyl methacrylate, methyl acrylate, ethyl acrylate, propyl acrylate, butyl acrylate, amyl acrylate, ethylhexyl acrylate, octyl acrylate, t-octyl acrylate, 2-methoxyethyl acrylate, 2-butoxyethyl acrylate, 2-phenoxyethyl acrylate, chloroethyl acrylate, cyanoethyl acrylate, dimethylaminoethyl acrylate, benzyl acrylate, methoxybenzyl acrylate, furfuryl acrylate, tetrahydrofurfuryl acrylate and phenyl acrylate; di(meth)acrylates may be selected from 2,2-bis(4-methacryloxyphenyl)propane, 1,2-butanediol-diacrylate, 1,4-butanediol-diacrylate, 1,4-butanediol-dimethacrylate, 1,4-cyclohexanediol-dimethacrylate, 1,10-decanediol-dimethacrylate, diethylene-glycol-diacrylate, dipropyleneglycol-diacrylate, dimethyl-propanediol-dimethacrylate, triethyleneglycol-dimethacrylate, tetraethyleneglycol-dimethacrylate, 1,6-hexanediol-diacrylate, neopentylglycol-diacrylate, polyethyleneglycol-dimethacrylate, tripropyleneglycol-diacrylate, 2,2-bis[4-(2-acryloxyethoxy)-phenyl]propane, 2,2-bis[4-(2-hydroxy-3-methacryloxypropoxy)-phenyl]propane, bis(2-methacryloxyethyl)N,N-1,9-nonylene-biscarbamate, 1,4-cycloheanedimethanol-dimethacrylate, and diacrylic urethane oligomers; tri(meth)acrylates may be selected from tris(2-hydroxyethyl)-isocyanurate-trimethacrylate, tris(2-hydroxyethyl)isocyanurate-triacrylate, trimethylolpropane-trimethacrylate, trimethylolpropane-triacrylate or pentaerythritol-triacrylate; tetra(meth)acrylates may be selected from pentaerythritol-tetraacrylate, di-trimethylopropan-tetraacrylate, or ethoxylated pentaerythritol-tetraacrylate; suitable penta(meth)acrylates may be selected from dipentaerythritol-pentaacrylate or pentaacrylate-esters; examples for polyacrylates are polyisobornylacrylate, polyisobornylmethacrylate, polyethoxyethoxyethylacrylate, poly-2-carboxyethylacrylate, polyethylhexylacrylate, poly-2-hydroxyethylacrylate, poly-2-phenoxylethylacrylate, poly-2-phenoxyethylmethacrylate, poly-2-ethylbutylmethacrylate, poly-9-anthracenylmethyl methacrylate, poly-4-chlorophenylacrylate, polycyclohexylacrylate, polydicyclopentenyl-oxyethylacrylate, poly-2-(N,N-diethylamino)ethylmethacrylate, poly-dimethylamino-eopentylacrylate, poly-caprolactone 2-(methacryloxy)ethylester, polyfurfurylmethacrylate, poly(ethylene glycol)methacrylate, polyacrylic acid and poly(propylene glycol)methacrylate, as well as mixtures, copolymers and combinations of any of the foregoing.

Suitable polyacrylates may also comprise aliphatic unsaturated organic compounds, e.g., polyacrylamide and unsaturated polyesters from condensation reactions of unsaturated dicarboxylic acids and diols, as well as vinyl-derivatives, or compounds having terminal double bonds, e.g., N-vinylpyrrollidone, styrene, vinyl-naphthalene or vinylphtalimide. Methacrylamid-derivatives that may be used include N-alkyl- or N-alkylen-substituted or unsubstituted (meth)acrylamide, such as acrylamid, methacrylamide, N-methacrylamide, N-methylmethacrylamide, N-ethylacrylamide, N,N-dimethylacrylamide, N,N-dimethylmethacrylamide, N,N-diethylacrylamide, N-ethylmethacrylamide, N-methyl-N-ethylacrylamide, N-isopropylacrylamide, N-n-propylacrylamide, N-isopropylmethacrylamide, N-n-propylmethacrylamide, N-acryloyloylpyrrolidine, N-methacryloylpyrrolidine, N-acryloylpiperidine, N-methacryloylpiperidine, N-acryloylhexahydroazepine, N-acryloylmorpholine or N-methacryloylmorpholine.

Further suitable polymers that may be used as the matrix material in accordance with exemplary embodiments of the present invention can include unsaturated and saturated polyesters, and alkyd resins. The polyesters may contain polymeric chains, a varying number of saturated or aromatic dibasic acids and anhydrides, or epoxy resins, which may be provided as monomers, oligomers or polymers, particularly those which comprise one or several oxirane rings, one aliphatic, aromatic or mixed aliphatic-aromatic molecular structural element, or exclusively non-benzoid structures, e.g., aliphatic or cycloaliphatic structures with or without substituents such as halogen, ester groups, ether groups, sulfonate groups, siloxane groups, nitro groups, or phosphate groups, or any combination thereof.

In further exemplary embodiments of the present invention, the matrix material may include epoxy resins, for example of the glycidyl-epoxy type, such as those equipped with diglycidyl groups of bisphenol A. Further epoxy resins which may be used include amino derivatized epoxy resins, particularly tetraglycidyl diaminodiphenyl methane, triglycidyl-p-aminophenol, triglycidyl-m-maminophenole, or triglycidyl aminocresole and their isomers, phenol derivatized epoxy resins such as, for example, epoxy resins of bisphenol A, bisphenol F, bisphenol S, phenol-novolac, cresole-novolac or resorcinole, phenoxy resins, as well as alicyclic epoxy resins. Furthermore, halogenated epoxy resins, glycidyl ethers of polyhydric phenols, diglycidylether of bisphenol A, glycidylethers of phenole-formaldehyde-novolac resins and resorcinole diglycidylether, as well as epoxy resins such as those described, for example, in U.S. Pat. No. 3,018,262. These materials may be solidified or cured thermally or by applying radiation or cross linking techniques.

Epoxy resins can be particularly preferred for use as matrix materials in combination with reticulating agents that can include metal or metal oxide particles or combinations thereof. Epoxy resins may also be used in combination with reticulating agents that include carbon particles and/or fullerenes.

In exemplary embodiments of the present invention, the matrix material may be substantially non-elastic, and/or it may be substantially free of fibers or particles.

Suitable matrix materials are not restricted to the materials mentioned above. For example, mixtures of epoxy resins that include two or several components as described above may be used, as well as monoepoxy components. The epoxy resins may also include resins that can be cross linked using radiation, e.g., ultraviolet (UV) radiation, and cycloaliphatic resins.

Further suitable matrix materials may include polyamides such as, e.g., aliphatic or aromatic polyamides and aramides (e.g., nomex®), and their derivatives, e.g., nylon-6-(polycaprolactam), nylon 6/6 (polyhexamethyleneadipamide), nylon 6/10, nylon 6/12, nylon 6/T (polyhexamethylene terephthalamide), nylon 7 (polyenanthamide), nylon 8 (polycapryllactam), nylon 9 (polypelargonamide), nylon 10, nylon 11, nylon 12, nylon 55, nylon XD6 (poly metha-xylylene adipamide), nylon 6/1, and poly-alanine.

Other suitable matrix materials may include, for example, metal phosphinates or polymetal phosphinates as well as inorganic metal-containing polymers or organic metal-containing polymers such as, for example, metallodendrimers, metallocenyl polymers, carbosilanes, polyynes, noble metal alkynyl polymers, metalloporphyrine polymers, metallocenophanes, metallocenylsilane-carbosilane copolymers as mono, diblock, triblock or multiblock copolymers, or poly(metallocenyldimethylsilane) compounds, carbothiametallocenophanes, poly(carbothiametallocenes) and the like, or any combinations thereof.

In an exemplary embodiment of the present invention, the matrix material may include electrically conducting polymers, such as saturated or unsaturated polyparaphenylene-vinylene, polyparaphenylene, polyaniline, polythiophene, poly(ethylenedioxythiophene), polydialkylfluorene, polyazine, polyfurane, polypyrrole, polyselenophene, poly-p-phenylene sulfide, polyacetylene, and monomers, oligomers or polymers or any combinations and mixtures thereof with other monomers, oligomers or polymers or copolymers that include the above-mentioned monomers. The conductive or semi-conductive polymers may have an electrical resistance between about 10⁻¹² and 10¹² Ohm-cm, and may comprise monomers, oligomers or polymers which include one or several organic radicals, for example, alkyl- or aryl-radicals and the like, or inorganic radicals, such as silicone or germanium and the like, or any mixtures thereof.

Polymers which can include complexed metal salts may also be used to provide the matrix material. Such polymers can comprise an oxygen, nitrogen, sulfur or halogen atom or unsaturated C—C bonds, and they may be capable of complexing metals. Examples of such polymers can include elastomers such as, e.g., polyurethane, rubber, adhesive polymers and thermoplastics. Metal salts that can be used include, e.g., transition metal salts such as CuCl₂, CuBr₂, CoCl₂, ZnCl₂, NiCl₂, FeCl₂, FeBr₂, FeBr₃, CuI₂, FeCl₃, FeI₃, or FeI₂; or salts such as, e.g., Cu(NO₃)₂, metal lactates, glutamates, succinates, tartrates, phosphates, oxalates, LiBF₄, and H₄Fe(CN)₆ and the like.

In exemplary embodiments of the present invention, the matrix material may include biopolymers, bio-compatible or biodegradable polymers such as collagen, albumin, gelatin, hyaluronic acid, starch, celluloses such as methylcellulose, hydroxypropyl cellulose, hydroxypropyl methylcellulose, carboxymethylcellulose phthalate; casein, dextranes, polysaccharides, fibrinogen, poly(D,L-lactides), poly(D,L-lactide coglycolides), poly(glycolides), poly(hydroxybutylates), poly(alkylcarbonates), poly(orthoesters), poly(hydroxyvaleric acid), polydioxanones, poly(ethyleneterephthalates), poly(maleic acid), poly(tartaric acid), polyanhydrides, polyphosphazenes, poly(amino acids), or shellac.

The matrix material may include oligomers or elastomers such as, for example, polybutadiene, polyisobutylene, polyisoprene, poly(styrene-butadiene-styrene), polyurethanes, polychloroprene, or silicone, and any mixtures, copolymers and combinations thereof. The matrix material may also comprise pH-sensitive polymers such as, for example, poly(acrylic acid) and its derivatives, for example homopolymers such as poly(aminocarboxyl acid), poly(acrylic acid), poly(methyl-acrylic acid) and copolymers thereof; or temperature-sensitive polymers, such as, for example poly(N-isopropylacrylamide-Co-sodium-acrylate-Co-n-N-alkylacrylamide), poly(N-methyl-N-n-propylacrylamide), poly(N-methyl-N-isopropylacrylamide), poly(N-n-propylmethacrylamide), poly(N-isopropylacrylamide), poly(N,n-diethylacrylamide), poly(N-isopropylmethacrylamide), poly(N-cyclopropylacrylamide), poly(N-ethylacrylamide), poly(N-ethylmethyacrylamide), poly(N-methyl-N-ethylacrylamide), poly(N-cyclopropylacrylamide). Furthermore, suitable matrix material polymers having a thermogel characteristic which may be used include hydroxypropyl-cellulose, methylcellulose, hydroxylpropylmethyl-cellulose, ethylhydroxyethyl-cellulose and pluronics® such as, e.g., F-127, L-122, L-92, L81, or L61.

The matrix material may be provided in a liquid form during the manufacturing process used to form the medical device, e.g., as a liquid prepolymer, a melt, polymer or a solution, dispersion, or emulsion, and it may be mixed with one or more reticulating agents in the absence or presence of a solvent, or it may be provided in a solid form.

To produce a medical device in accordance with exemplary embodiments of the present invention, the reticulating agents can be combined with the matrix material, optionally in the presence or absence of a suitable solvent or solvent mixture, to form a mixture capable of flowing, e.g., a solution, suspension, dispersion or emulsion, or a melt, slurry, paste or flowable particle mixture. The liquid mixture may be substantially uniform and/or substantially homogenous. However, uniformity or homogeneity of the liquid mixture may not be required or important to form such medical devices.

Suitable solvents may comprise water, sols or gels, or nonpolar or polar solvents such as, e.g., methanol, ethanol, n-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, methylethyl ketone, ethoxypropylacetate, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofurane, trimethyl hexanol, phenol, benzene, toluene, or xylene, any of which may be mixed with dispersants, surfactants or other additives, or mixtures of the above-named substances.

Solvents that may be readily removable, e.g., easily volatized, may be used. These solvents may have a boiling point below 120° C., below 80° C., or optionally below 50° C. The solvent or solvent mixture can be used to facilitate effective dispersion of the solids, especially where uniform or homogenous liquid mixtures are preferred.

The solvent used in certain exemplary embodiments of the present invention may comprise solvent mixtures that may be suitable for dissolving or swelling the matrix material or at least a portion thereof if, e.g., the matrix material is a composite or mixture. Solvents that can substantially or completely dissolve the matrix material may be preferred in exemplary embodiments of the present invention.

In accordance with exemplary embodiments of the present invention, the liquid mixture may be in the form of a colloidal solution, a solid solution, a dispersion, a suspension or an emulsion, which comprises at least one matrix material and at least one reticulating agent. The matrix material, the reticulating agent, the solvent and optional additives may be selected in order to provide, for example, an essentially stable and optionally homogeneous dispersion, suspension, emulsion or solution.

The dynamic viscosity of the liquid mixture, e.g., a solution, dispersion, suspension or emulsion comprising a solvent, a matrix material and a reticulated agent, can be at least about 10 to 99%, preferably about 20 to 90%, or between about 50 to 90% below the viscosity of the matrix material at the application temperature of the liquid mixture before solidifying, which may preferably be about 25° C.

If the mixture capable of flowing does not include a solvent, the temperature and/or composition of the liquid mixture or the matrix material can be selected such that the dynamic viscosity of the mixture capable of flowing, free of any solvent, may be at least about 10% to 99%, preferably about 20% to 90% or about 50% to 90% below the viscosity of the matrix material at the temperature. These percentage values can be understood to refer to the mixture before any significant cross linking occurs and/or before cross linkers are added. Viscosities may be measured by conventional methods, e.g., in a capillary viscometer or using a Brookfield apparatus.

The combination of reticulating agents, the solvent and the matrix material can be selected such that the solvent, the matrix material and/or the liquid mixture wets the selected reticulating agents. Optionally, the reticulating agents may be modified by using suitable additives or surface modifiers, such as those described herein above, to increase their wettability, or optionally to be essentially fully wetted.

The reticulating agent and the matrix material may be combined in a specific weight or volume ratio, e.g., to optimize the structure of the porous composite formed under the conditions used for solidifying the liquid mixture. The specific ratio used may depend on the molecular weight, the particle size and the specific surface area of the particles. The ratio used can be selected such that upon removal of the solvent during the solidification procedure or upon changing the viscosity of the matrix component, a phase separation into a solvent phase and a solid phase comprising the matrix material and the reticulating agent can be achieved. The viscosity change can be achieved, e.g., by changing the temperature to higher or lower values, or by the addition of cross linkers, particularly in solvent free systems.

This phase separation can facilitate the formation of a three-dimensional network of the solid phase, e.g., by self-orientation of the components used. In exemplary embodiments of the present invention, the ratio of the total volume of the reticulating agents and the total volume of the matrix material can range from about 20:80 to 70:30, preferably from about 30:70 to 60:40, or from about 50:50 to 60:40.

In exemplary embodiments of the present invention, the solids content in the liquid mixture may be up to about 90% by weight of the total weight of the liquid mixture, preferably up to about 80%, or below about 20% by weight, preferably below about 15% by weight, e.g., below about 10% by weight or optionally below about 5% by weight.

Mechanical, optical and thermal properties of the composite material may be further adjusted and/or varied through the use of additives, which may be particularly suitable for producing tailor-made coatings. Thus, in certain exemplary embodiments of the present invention, further additives can be added to the liquid mixture.

Examples of suitable additives include, e.g., fillers, further pore-forming agents, metals and metal powders, etc. Examples of inorganic additives and fillers include silicon oxides and aluminum oxides, aluminosilicates, zeolites, zirconium oxides, titanium oxides, talc, graphite, carbon black, fullerenes, clay materials, phyllosilicates, silicides, nitrides, metal powders, including transition metals such as copper, gold, silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum.

Other suitable additives can include cross linkers, plasticizers, lubricants, flame resistants, glass or glass fibers, carbon fibers, cotton, fabrics, metal powders, metal compounds, silicon, silicon oxides, zeolites, titan oxides, zirconium oxides, aluminum oxides, aluminum silicates, talcum, graphite, soot, phyllosilicates and the like.

Additives that may be used to facilitate cross linking may include, e.g., organosilanes such as tetraalkoxysilanes, alkylalkoxysilanes, and aryltrialkoxysilanes such as those described herein above, or those described, e.g., in International Patent Application No. PCT/EP2006/050622 and U.S. patent application Ser. No. 11/346,983, filed Feb. 3, 2006.

Further additives may be added, e.g., for wetting, dispersing and/or sterically stabilizing the components of the mixture, or electrostatic stabilizers, rheology or thixotropy modifiers, such as the various additives and dispersing aids sold under the trademarks Byk®, Disperbyk® or Nanobyk® by Byk-Chemie GmbH, Germany, or equivalent compositions from other manufacturers.

Emulsifiers may also be included in the liquid mixture. Suitable emulsifiers may include, for example, anionic, cationic, zwitter-ionic or non-ionic surfactants or any combinations thereof. Anionic surfactants can include soaps, alkylbenzolsulfonates, alkansulfonates such as sodium dodecylsulfonate (SDS) and the like, olefinsulfonates, alkyethersulfonates, glycerinethersulfonates, α-methylestersulfonates, sulfonated fatty acids, alkylsulfates, fatty alcohol ether sulfates, glycerine ether sulfates, fatty acid ether sulfates, hydroxyl mixed ether sulfates, monoglyceride(ether)sulfates, fatty acid amide(ether)sulfates, mono- and di-alkylsulfosuccinates, mono- and dialkylsulfosuccinamates, sulfotriglycerides, amidsoaps, ethercarboxylicacid and their salts, fatty acid isothionates, fatty acid arcosinates, fatty acid taurides, N-acylaminoacids such as acyllactylates, acyltartrates, acylglutamates and acylaspartates, alkyoligoglucosidsulfates, protein fatty acid condensates, including plant-derived products based on wheat; and alky(ether)phosphates.

Suitable cationic surfactants may include quaternary ammonium compounds such as dimethyldistearylammoniumchloride, Stepantex® VL 90 (Stepan), esterquats such as quaternized fatty acid trialkanolaminester salts, salts of long-chain primary amines, quaternary ammonium compounds such as hexadecyltrimethyl-ammoniumchloride (CTMA-Cl), Dehyquart® A (cetrimoniumchloride, available from Cognis), or Dehyquart® LDB 50 (lauryldimethylbenzylammoniumchloride, available from Cognis).

Several additives may optionally be used to produce a stable dispersion, suspension or emulsion in the liquid mixture.

Additional fillers can be used to further modify the size and/or the degree of porosity. In exemplary embodiments of the present invention, non-polymeric fillers may be used. Non-polymeric fillers can include compositions that can be removed or degraded, for example, by thermal treatment, washing out, or other techniques, without adversely affecting the material properties. Some fillers can be dissolvable in a suitable solvent and may be removed in this manner from the final material. Furthermore, non-polymeric fillers, which can be converted into soluble substances under certain thermal conditions, can also be used. Non-polymeric fillers may include, for example, anionic, cationic or non-ionic surfactants, which can be removed and/or degraded under certain thermal conditions. Fillers can also include inorganic metal salts, particularly salts from alkaline and/or alkaline earth metals, such as alkaline or alkaline earth metal carbonates, sulfates, sulfites, nitrates, nitrites, phosphates, phosphites, halides, sulfides, and oxides. Other suitable fillers can comprise organic metal salts, e.g. alkaline or alkaline earth and/or transition metal salts, their formiates, acetates, propionates, malates, maleates, oxalates, tartrates, citrates, benzoates, salicylates, phthalates, stearates, phenolates, sulfonates, and amines, as well as mixtures thereof.

In another exemplary embodiment of the present invention, polymeric fillers can be used. Suitable polymeric fillers can include those mentioned herein above as encapsulation polymers, particularly if provided in the form of spheres or capsules. Certain examples may include saturated, linear or branched aliphatic hydrocarbons, which can be homo- or copolymers, e.g. polyolefins such as polyethylene, polypropylene, polybutene, polyisobutene, polypentene as well as copolymers and mixtures thereof. Furthermore, polymer particles formed of methacrylates or polystearine, as well as electrically conducting polymers as described herein above, e.g. polyacetylenes, polyanilines, poly(ethylenedioxythiophenes), polydialkylfluorenes, polythiophenes or polypyrroles, can also be used as polymeric fillers, e.g., for providing electrically conductive materials.

In the above-mentioned procedures, soluble fillers and polymeric fillers can be combined, where these fillers may be volatile under the thermal conditions used, e.g., in a solidification process, or they can be converted into volatile compounds during a thermal treatment. In this manner, pores formed by the polymeric fillers can be combined with pores formed by the reticulating agents and/or other fillers to achieve an isotropic or anisotropic pore distribution such as, for example, a hierarchical pore size distribution.

Suitable particle sizes of the non-polymeric fillers can be selcted based on the desired porosity and/or size of the pores of the resulting composite material.

Solvents which may be suitable for removal of the fillers or for cleaning procedures after solidification of the material may include, for example, water, hot water, diluted or concentrated inorganic or organic acids, bases, or any of the solvents mentioned herein above. Suitable inorganic acids may include, for example, hydrochloric acid, sulfuric acid, phosphoric acid, nitric acid, or diluted hydrofluoric acid. Suitable bases may include, for example, sodium hydroxide, ammonia, carbonate, or organic amines. Suitable organic acids may include, for example, formic acid, acetic acid, trichloromethane acid, trifluoromethane acid, citric acid, tartaric acid, oxalic acid, and mixtures thereof.

Fillers can be partly or completely removed from the reticulated composite material, depending on the nature and time of treatment with the solvent. Complete removal of the filler after solidification may be preferred in certain applications.

The solidification procedure selected may depend on specific properties and composition of the liquid mixture used. Solidification may be achieved, e.g., by thermal treatment such as heating or cooling, variation of pressure, e.g. evacuation, flushing or ventilation, drying with gases, including inert gases, drying, freeze-drying, spray-drying, filtration, or chemical or physical curing or hardening, e.g., with the use of cross linkers, optionally combined with a thermal cross linking or radiation-induced cross linking, or any combinations thereof.

Solidification may occur without significant decomposition of the matrix material or the combination of the reticulating agent and matrix material, e.g., without significant thermolysis or pyrolysis of the matrix material.

Suitable solidification conditions such as temperature, atmosphere or pressure, can be selected to provide a substantially complete solidification based on the desired properties of the composite material being formed and the components used.

In additional exemplary embodiments of the present invention, the solidification procedure may include a phase separation of the liquid mixture into a solid phase and a liquid phase, e.g., by precipitating the solids from the liquid mixture. Such a phase separation or precipitation may facilitate or promote the development of a reticulated structure in the resulting composite material. The structural development may occur substantially before the solvents are removed, e.g., the phase separation or precipitation may be induced before removal of the at least one solvent. The phase separation or precipitation can be induced by removal of solvent, cross linking of the matrix material, and/or increasing the viscosity of the liquid mixture.

The increase in viscosity of the liquid mixture may be provided by, e.g., cross linking, curing, drying, rapidly increasing the temperature, rapidly lowering the temperature, or rapidly removing the solvent. “Rapidly” can refer to a time period less than about 5 hours, preferably less than about one hour, or within less than about 30 minutes, 20 minutes, 15 minutes, 10 minutes, 5 minutes or less than about 2 minutes or less than about 1 minute after increasing of the viscosity is initiated. The time period required may depend on the mass of the liquid mixture being processed.

A thermal treatment may include heating or cooling in a temperature range between about −78° C. to 500° C., and may include a heating process, a freezing process, a freeze-drying process, and the like.

The solvent can be removed from the liquid mixture prior to a thermal treatment. This can be achieved by filtration, or alternatively by a thermal treatment of the liquid mixture, e.g., by cooling or heating the liquid mixture to temperatures between about −200° C. to 300° C., between about −100° C. to 200° C., or between about −50° C. to 150° C. Temperatures between about 0° C. to 100° C. or about 50° C. to 80° C. may also be used. An evaporation of the solvents at room temperature or in a stream of hot air or other gases can also be used. Drying may be performed by spray drying, freeze-drying, or similar conventional methods.

The solidification treatment may also include a thermal treatment at elevated temperatures, with or without prior removal of the solvent, which may be performed at temperatures between about 20° C. to 4000° C., or between about 100° C. to 3500° C. Temperatures between about 100° C. and 2000° C. or between about 150° C. and 500° C. may also be used. The thermal treatment can optionally be performed under reduced pressure or vacuum, or in the presence of inert or reactive gases.

A solidification process that does not decompose any of the components can be performed at temperatures up to about 500° C. In certain exemplary embodiments of the present invention, it may be preferred to partially or totally carbonize, pyrolize or decompose at least one of the components of the composite material during or after the solidification process. This can be achieved at higher temperatures, which may range from about 150° C. to about 4000° C. These higher temperatures can be used in further exemplary embodiments of the present invention where an additional sintering step may be desired.

Sintering procedures at high temperatures, e.g., temperatures above 500° C., may not be required or desired when forming certain composite materials, and treatment steps involving decomposition of matter, e.g., pyrolysis or carbonization steps, may preferably be avoided. The solidification procedure that may be performed in accordance with certain exemplary embodiments of the present invention may be performed at temperatures between about 20° C. and 500° C., or between about 30° C. and 350° C. Temperatures between about 40° C. and 300° C., or below about 200° C., e.g., between about 100° C. and 190° C., may also be used.

The solidification procedure can be performed in different atmospheres. For example, it can be performed in an inert atmosphere such as, for example, nitrogen, SF₆, or a noble gas such as argon, or any mixtures thereof. The solidification procedure may also be performed in an oxidizing atmosphere comprising, e.g., oxygen, carbon monoxide, carbon dioxide, or nitrogen oxide. Furthermore, an inert atmosphere can be blended with reactive gases, e.g., hydrogen, ammonia, C₁-C₆ saturated aliphatic hydrocarbons such as methane, ethane, propane and butane, or mixtures thereof.

In certain exemplary embodiments of the present invention, the atmosphere in the solidification procedure, particularly when thermally treating the liquid mixture, can be an oxidizing atmosphere such as air, oxygen or oxygen enriched inert gases. Alternatively, the atmosphere during the solidification procedure can be substantially free of oxygen, e.g., the oxygen content may be below about 10 ppm, or even below about 1 ppm.

The solidification process can also be performed using laser applications, e.g., by using a selective laser sintering (SLS) technique, and/or may be radiation-induced, e.g., when using UV or gamma radiation to activate and cure cross linkers that can be present in the mixture.

Solid components can be precipitated from a solvent-based liquid mixture by thermal treatment, by cross linking, and/or by evaporating the solvent. A low viscosity of the liquid mixture may be preferred when forming a substantially homogeneous porous structure in the resulting composite material and/or to promote a network-like or reticulated orientation of particles in the liquid mixture, which may be accompanied by a rapid viscosity increase of the solid phase during the solidification procedure. This can be achieved by separating the solid phase from the solvent phase, where the temperature for this process may be selected based on the freezing point or the boiling point, respectively, of the solvent and the matrix material.

If solidification is achieved by increasing the temperature of the mixture, the solvent may have a boiling point that is lower than the melting point of the matrix material by at least about 5° C. and up to about 200° C., between about 30° C. and 200° C. lower, or between about 40° C. and 100° C. below the melting point of the matrix material, so that there may be little or no reduction of the viscosity of the matrix material, and/or no melting or incomplete thermal decomposition of the matrix material or the reticulating agents during thermal treatment of the liquid mixture and/or during removal of the solvent.

In a preferred exemplary embodiment of the present invention, a rapid lowering of the temperature may be used to solidify the liquid mixture. This can be achieved with liquid mixtures that may or may not include a solvent. In a solvent-based mixture, the solvent may have a boiling point that is at least 10° C. to 100° C. above the melting point of the matrix material, preferably about 20° C. to 100° C. higher, or more preferably about 30° C. to 60° C. above the melting point of the matrix material.

By providing a dispersion, suspension, emulsion or solution at a temperature close to that of the melting point of the matrix material, which may be a polymer, a network of the reticulating agents may be formed by rapidly lowering the temperature, resulting in a rapid increase of the viscosity of the liquid mixture. To incorporate the reticulating agents in the matrix material, the solvent phase can subsequently be removed from the liquid mixture by using, e.g., a vacuum treatment.

Cross linkers can be added to a dispersion, suspension or emulsion that may be provided as the liquid mixture. Cross linkers may include, for example, isocyanates, silanes, diols, di-carboxylic acids, (meth)acrylates, for example such as 2-hydroxyethyl methacrylate, propyltrimethoxysilane, 3-(trimethylsilyl)propyl methacrylate, isophoron diisocyanate, polyols, glycerin and the like. Biocompatible cross linkers such as glycerin, diethylentriaminoisocyanate and 1,6-diisocyanatohexane, may be preferred, e.g., if the liquid mixture is to be converted into the solid composite material at relatively low temperatures, e.g., below about 100° C.

The composition and type of the cross linker can be selected such that the cross linking that may occur during solidification of the liquid mixture does not generate a significant viscosity change of the system before the solid composite phase has formed by phase separation or evaporation of the solvent. Cross linking may be interrupted, and components of the matrix material which are not already cross linked or only partially cross linked may be dissolved and removed by treating the system with suitable solvents, in order to modify the morphology and the overall structure of the composite material.

The liquid mixture and/or the formed composite material that may be used to form or coat the medical device may be subjected to further processing, depending on the particular intended use. For example, reductive or oxidative treatment procedures can be applied in which the solidified material or coating may be treated one or more times with suitable reducing agents and/or oxidizing agents such as, e.g., hydrogen, carbon dioxide, water vapor, oxygen, air, or nitrous oxide, or oxidizing acids such as nitric acid and the like, and optionally mixtures of these agents, to modify pore sizes and/or surface properties. Activation of surface properties with air may also be performed. This can be achieved at an elevated temperature, e.g., between about 40° C. and 1000° C., or between about 70° C. and 900° C., or between about 100° C. and 850° C. Temperatures between, about 200° C. and 800° C. may also be used, or the selected temperature may be approximately 700° C. The composite material produced in accordance with exemplary embodiments of the present invention can also be modified by reduction or oxidation, or by a combination of these treatment steps at approximately room temperature. Boiling of the composite material in oxidizing acids or bases may also be used to modify surface and bulk properties.

The pore size and/or pore structure of the composite material formed can be varied according to the type of oxidizing agent or reducing agent used, the activation temperature selected, and/or the duration of the activation. The porosity can be adjusted by, e.g., washing out fillers that may be present in the composite material, as described above. These fillers can include polyvinylpyrrolidone, polyethylene glycol, powdered aluminum, fatty acids, microwaxes or emulsions thereof, paraffins, carbonates, dissolved gases or water-soluble salts, which may be removed, e.g., by treatment of the material with water, solvents, acids or bases, or by distillation or oxidative and/or non-oxidative thermal decomposition. Suitable treatment methods to achieve removal of fillers are described, for example, in German Patent Publication No. DE 103 22 187 and International Patent Application No. PCT/EP2004/005277.

The properties of the composite material produced may optionally be altered by structuring the surface with powdered substances such as, e.g., metal powder, carbon black, phenolic resin powder or fibers, including carbon fibers or natural fibers.

The composite material may also be optionally subjected to a chemical vapor deposition (CVD) process or a chemical vapor infiltration (CVI) process to further modify the surface structure and/or pore structure and/or its physico-chemical properties. This can be achieved by treating the composite material or coating with suitable precursor gases that can release carbon at high temperatures. For example, a diamond-like carbon coating or film can be applied using these techniques. Other elements, such as silicon, may also be deposited onto or into the composite material using these conventional techniques. Many saturated and unsaturated hydrocarbons having sufficient volatility under CVD conditions may be used as a precursor to split off carbon. Suitable ceramic precursors include, for example, BCl₃, NH₃, silanes such as SiH₄, tetraethoxysilane (TEOS), dichlorodimethylsilane (DDS), methyltrichlorosilane (MTS), trichlorosilyldichloroborane (TDADB), hexadichloromethylsilyloxide (HDMSO), AlCl₃, TiCl₃ or mixtures thereof. Using CVD techniques, the size of pores in the material can be reduced in a controlled manner, or the pores may be completely closed and/or sealed. This exemplary process can allow controlled adjustment or variation of sorptive properties and/or mechanical properties of the composite material. The composite materials or coatings can be modified by a CVD process using silanes or siloxanes, optionally in a mixture with hydrocarbons, to form a carbide or oxycarbide, which can achieve increased resistance of the material to oxidation.

The materials or devices produced according to exemplary embodiments of the present invention can be further coated and/or modified using sputtering techniques or ion implantation/ion bombardment techniques. Carbon, silicon, metals and/or metal compounds can be applied using conventional techniques and suitable sputter targets. For example, by incorporating silicon compounds, titanium compounds, zirconium compounds, tantalum compounds or metals into the material using CVD or PVD techniques, carbide phases can be formed which may increase the stability and oxidation resistance of the material.

The composite materials as described herein may have an average pore size that is larger than about 1 nm, preferably larger than about 5 nm, more preferably larger than about 10 nm or larger than about 100 nm. Pore sizes may range from between about 1 nm to 400 μm, preferably between about 1 nm to 80 μm, or more preferably about 1 nm to 40 μm. Pore sizes in a macro porous material may range from between about 500 nm to 1000 μm, preferably from about 500 nm to 800 μm, 500 nm to 500 μm, or about 500 nm to 80 μm. An average porosity of the material can be between about 30% and 80%.

The composite material can also be worked mechanically to produce porous surfaces. For example, controlled abrasion of the surface layer(s) by suitable methods can lead to modified porous surface layers. One technique that may be used to achieve this can include cleaning and/or abrasion in an ultrasonic bath, where defects in the material and further porosity can be produced in a targeted manner by admixture of abrasive solids of various particle sizes and degrees of hardness, and by appropriate input of energy and a suitable frequency of the ultrasonic bath as a function of treatment time. Aqueous ultrasonic baths, to which alumina, silicates, aluminates or the like have been added, preferably alumina dispersions, may be used. Any solvent that is suitable for ultrasonic baths may also be used instead of or in combination with water.

Ion implantation of metal ions, in particular transition metal ions and/or non-metal ions, can be used to further modify the surface properties of the composite material. For example, nitrogen implantation may be used to incorporate nitrides, oxynitrides or carbonitrides, in particular those of the transition metals. The porosity and strength of the surface of the materials can be further modified by implantation of carbon.

The composite materials can be further modified, e.g., by applying biodegradable, resorbable and/or non-biodegradable resorbable polymers, which optionally may be provided in a porous form, as a layer form or an overcoat onto the composite material.

Parylenation of the medical devices provided in accordance with exemplary embodiments of the present invention may be performed before or after any activation procedure(s) described herein to further modify their surface properties and/or porosity. A device can first be treated with para-cyclophane at an elevated temperature, which may be approximately 600° C., to form a polymer film of poly(p-xylylene) on the surface of the device. This film can optionally be converted to carbon using a subsequent conventional carbonization step.

If desired, the composite material may be subjected to additional chemical and/or physical surface modifications. Cleaning steps to remove residues and/or impurities that might be present can be performed on the composite material. For example, acids such as oxidizing acids, or solvents may be used, but boiling in acids or solvents may be used. Carboxylation of certain materials can be achieved by boiling in oxidizing acids. Washing with organic solvents, optionally combined with application of ultrasound energy, and optionally performed at elevated temperatures, may also be used for further processing of the devices.

The composite materials and devices formed therefrom may be sterilized using conventional methods, e.g., by autoclaving, ethylene oxide sterilization, pressure sterilization or gamma-radiation. The various modification and cleaning procedures described herein may be performed in any combination, depending on the components of the material formed and the desired properties.

Coatings or bulk compositions comprising the porous composite material which may be provided in and/or on the medical devices may be structured in a suitable manner, before or after solidification, using such techniques as, for example, folding, embossing, punching, pressing, extruding, gathering, injection molding and the like. These techniques may be applied before or after the composite material is applied to the substrate or molded or formed. In this way, certain structures of a regular or irregular type can be incorporated, for example, into a coating produced using the composite material provided according to exemplary embodiments of the present invention. The composite material can be further processed using conventional techniques to form the desired medical devices, or a portion thereof, e.g., by providing molded paddings and the like, or by forming coatings on existing medical devices.

The medical devices can be produced in any desired form. By applying multi-layered half-finished molded shapes, asymmetric constructions can be formed from the composite materials. The materials can be brought into the desired form by applying any appropriate conventional technique, including but not limited to casting processes such as sand casting, shell molding, full mold processes, die casting and centrifugal casting, or by pressing, sintering, injection molding, compression molding, blow molding, extrusion, calendaring, fusion welding, pressure welding, jiggering, slip casting, dry pressing, drying, firing, filament winding, pultrusion, lamination, autoclave, curing or braiding.

Coatings of the composite material can be applied in a liquid, pulpy or pasty form, for example, by painting, furnishing, phase-inversion, dispersing atomizing or melt coating, extruding, die casting, slip casting, dipping or as a hot melt. Coatings may be applied directly in the form of the liquid mixture before solidification of the mixture is performed. If the coating material is already in a solid state, it may be applied on a suitable substrate by, e.g., powder coating, flame spraying, sintering or the like, to form the medical device. Techniques such as, for example, dipping, spraying, spin coating, ink-jet-printing, tampon and microdrop coating or 3-D-printing may also be used to apply the liquid mixture onto a substrate. The application of the liquid mixture can be performed using a high frequency atomizing device such as, for example, the one described in International Patent Application No. PCT/EP2005/000041, or by print or roller coating using a device such as the one described, e.g., in International Patent Publication No. WO 2005/042045. Such devices and techniques may also be used to coat the medical device with additional agents, e.g. therapeutically or diagnostically active agents, or with further coatings as described herein below. A coating of the composite material can be provided, for example, by applying a layer of the liquid mixture to a medical device, drying it, and optionally performing a thermal treatment.

Coated devices can be provided by a transfer process, in which the composite material may be applied to a device substrate in the form of a prepared lamination. The coated device can be dried, cured and the coating may then optionally be treated or further processed. A coated medical device can also be obtained using suitable printing procedures, e.g., gravure printing, scraping or blade printing, spraying techniques, thermal laminations, or wet-in-wet laminations. More than one thin layer can be applied, for example, to produce an error-free composite film. By applying the above-mentioned transfer procedures, multi-layer gradient films may be formed from different layers and/or different sequences of layers which, after the solidification procedure is performed, can provide gradient materials where the density of the composite material may vary with location in the film.

The liquid mixture can also be dried or thermally treated and then ground or pulverized using conventional techniques, for example, by grinding in a ball mill or a roller mill and the like. The pulverized composite material can be provided, for example, as a powder, a flat blank, a rod, a sphere, or a hollow sphere in different grainings, and can be processed using conventional techniques into granulates or extrudates in various forms. Hot-pressure-procedures, optionally accompanied by suitable binders, can be used to form the medical device or parts thereof from the composite material.

Additional processing options may include, for example, the formation of powders by other conventional techniques such as spray-pyrolysis or precipitation, or the formation of fibers by spinning techniques such as gel spinning.

By suitably selecting the components and the processing conditions, medical devices can be produced in accordance with exemplary embodiments of the present invention having inherent, direct or indirect diagnostic and/or therapeutic effects, with bioerodible or biodegradable coatings, and/or with coatings of composite materials which may be dissolvable or which may be peeled off of the devices in the presence of physiologic fluids.

In an exemplary embodiment of the present invention, the medical device can comprise at least one active agent for therapeutic and/or diagnostic purposes. The therapeutically and/or diagnostically active agent may be included in the medical device as a component of the reticulating agent, the matrix material or an additive, or it may be applied onto or into the composite material of the medical device, e.g., after the solidification procedure is performed.

A diagnostically active agent may include, for example, a marker, a contrast medium or a radiopaque material, and may be formed at least in part from one or more materials having signaling properties, e.g., materials that can produce a signal detectable by physical, chemical or biological detection methods. Examples of such materials are mentioned above as reticulating agents. Further suitable diagnostic agents having signaling properties are described, for example, in U.S. patent application Ser. No. 11/322,694, filed Dec. 30, 2005, and International Patent Application No. PCT/EP2005/013732. Certain matrix materials may also have signaling properties and thus may be used to provide a marker or a contrast medium. The device may also be suitably modified, for example, to allow for a controlled release of the diagnostic agent. The terms “diagnostically active agent,” “diagnostic agent,” “agent for diagnostic purpose” and “marker” can refer to such diagnostically active agents as described herein above.

A coating which can be applied to coronary implants such as stents may be produced as described herein, wherein the coating comprises an encapsulated marker, e.g., a metal compound having signaling properties that is capable of producing signals detectable by physical, chemical or biological detection techniques such as, e.g., x-ray techniques, nuclear magnetic resonance (NMR) techniques, computer tomography techniques, scintigraphy techniques, single-photon-emission computed tomography (SPECT) techniques, ultrasonic techniques, radiofrequency (RF) techniques, and the like.

For example, metal-based reticulating agents which may be used as markers can be encapsulated in a polymer shell, and thus can be prevented from interfering with the medical device. The device can be made from an implant material, which may also comprise a metal, and interference between the two metal-containing compositions may lead to, e.g., electrocorrosion or similar undesirable effects. Coated implants may be produced which contain encapsulated markers, wherein the coating can remain on the implant permanently or for an extended period of time. In one exemplary embodiment of the present invention, the coating may be rapidly dissolved or peeled off from a stent after implantation of the stent under physiologic conditions, allowing a transient marking to occur.

Therapeutically active reticulating agents, if used, may be encapsulated in bioerodible or resorbable materials, which may optionally allow a controlled release of the active ingredient under physiologic conditions. Also, coatings or composite materials having a known and/or controlled porosity may be infiltrated or loaded with therapeutically active agents, which can be resolved or extracted in the presence of physiologic fluids. Thus, medical devices or implants may be provided which are capable of controllably releasing active agents. Examples of such devices include, and are not limited to drug eluting stents, drug delivery implants, drug eluting orthopedic implants and the like.

Medical devices in accordance with exemplary embodiments of the present invention may also be provided in the form of, e.g., porous bone or tissue grafts (erodible and non-erodible), porous implants or joint implants, or porous traumatologic devices such as nails, screws or plates. Such devices may optionally be coated, and further may be provided, e.g., with enhanced engraftment properties, therapeutic functionality, and/or excitable radiating properties, e.g., for performing local radiation therapy of tissues and organs.

Medical devices comprising composite materials and/or coatings can include conductive fibers such as, e.g., carbon nanotubes, which may reflect or absorb electromagnetic irradiation and therefore can provide shielding properties for electronic medical devices, such as metal implants, pacemakers or parts thereof.

Carbon nanotube- and nanofiber-based porous composite materials having high specific surface areas and particular thermal conductivity and/or anisotropic electrical conductivity can be produced for use, e.g., as actuators for micro- and macro-applications, or as thin film materials which can be used to make, e.g., artificial muscles or actuating fibers and films.

Medical devices in accordance with exemplary embodiments of the present invention may be further loaded with active ingredients. Active ingredients may be loaded into or onto the porous composite material using suitable sorptive methods such as adsorption, absorption, physisorption or chemisorption. Active agents may also be loaded by impregnating porous regions of the medical device with active ingredient solutions, active ingredient dispersions or active ingredient suspensions provided in suitable solvents. Active ingredients can be covalently or non-covalently bonded into or onto the medical device, depending on the active ingredient used and its chemical properties.

Active agents may be biologically and/or therapeutically active agents, or active agents that can be used for diagnostic purposes. Such active agents can include therapeutically active agents that are capable of providing direct or indirect therapeutic, physiologic and/or pharmacologic effects in a human or animal organism. A therapeutically active agent may be a drug, a pro-drug or even a targeting group or a drug comprising a targeting group.

The active agents may be provided in a crystalline, polymorphous or amorphous form or any combination thereof. Examples of therapeutically active agents include, but are not limited to, enzyme inhibitors, hormones, cytokines, growth factors, receptor ligands, antibodies, antigens, ion binding agents such as crown ethers and chelating compounds, substantially complementary nucleic acids, nucleic acid binding proteins including transcriptions factors, toxines and the like. Further examples of active agents that may be used in exemplary embodiments of the present invention include active agents, therapeutically active agents and drugs described, for example, in International Patent Application No. PCT/EP2006/050622 and U.S. patent application Ser. No. 11/346,983, filed Feb. 3, 2006.

Examples of active agents include, for example, cytokines such as erythropoietin (EPO), thrombopoietin (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine, and further active agents such as those described in Physician's Desk Reference, 58^{th} Edition, Medical Economics Data Production Company, Montvale, N.J., 2004 and the Merck Index, 13^{th} Edition, including those listed on pages Ther-1 to Ther-29.

In yet another exemplary embodiment of the present invention, the therapeutically active agent may be selected from the group of drugs used for the therapy of oncological diseases and cellular or tissue alterations. Suitable therapeutic agents can include, e.g., antineoplastic agents, including alkylating agents such as alkyl sulfonates, e.g., busulfan, improsulfan, piposulfane, aziridines such as benzodepa, carboquone, meturedepa, uredepa; ethyleneimine and methylmelamines such as altretamine, triethylene melamine, triethylene phosphoramide, triethylene thiophosphoramide, trimethylolmelamine; so-called nitrogen mustards such as chlorambucil, chlornaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, and the like, as well as combinations and/or derivatives of these agents.

In a further exemplary embodiment of the present invention, the therapeutically active agent may include anti-viral or anti-bacterial agents such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsäure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, and the like, as well as combinations and/or derivatives of any of these agents.

In a still further exemplary embodiment of the present invention, the therapeutically active agent may comprise radio-sensitizer drugs, steroidal or non-steroidal anti-inflammatory drugs, or agents referring to angiogenesis, such as, e.g., endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 and the like, as well as combinations and/or derivatives of any of these agents.

In another exemplary embodiment of the present invention, the therapeutically active agent may be selected from the group comprising nucleic acids, wherein the term nucleic acids may comprise oliogonucleotides having at least two nucleotides covalently linked to each other, for example, to provide gene therapeutic or antisense effects. Nucleic acids may further comprise phosphodiester bonds, which can include analogs having different backbones. Analogs may also contain backbones such as, for example, phosphoramide such as those described in, for example, Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986); phosphorothioate as described in, for example, Mag et al., Nucleic Acids Res. 19:1437 (1991) and in U.S. Pat. No. 5,644,048, phosphorodithioate as described in, for example, Briu et al., J. Am. Chem. Soc. 111:2321 (1989), O-methylphosphoroamidit-compounds (see, e.g., Eckstein, Oligonucleotides and Analogs: A Practical Approach, Oxford University Press), and peptide-nukleic acid-backbones and their compounds as described in, for example, Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996). Further analogs may include those having ionic backbones as described in, for example, Denpcy et al., Proc. Natl. Acad. Sci. USA 92:6097 (1995), or non-ionic backbones as described in, for example, U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research,” Ed. Y. S. Sanghui and P. Dan Cook. Nucleic acids having one or more carbocylic sugars may also be suitable as nucleic acids for use in exemplary embodiments of the present invention, such as those described in Jenkins et al., Chemical Society Review (1995), pages 169-176 and in Rawls, C & E News, 2 Jun. 1997, page 36. In addition to conventional nucleic acids and nucleic acid analogs, mixtures of naturally occurring nucleic acids and nucleic acid analogs or mixtures of nucleic acid analogs may also be used.

In a further exemplary embodiment of the present invention, the therapeutically active agent may comprise one or more metal ion complexes, such as those described in International Patent Application Nos. PCT/US95/16377, PCT/US95/16377, PCT/US96/19900, and PCT/US96/15527, where such agents may reduce or inactivate the bioactivity of their target molecules, including proteins such as enzymes.

Therapeutically active agents may also be anti-migratory, anti-proliferative or immune-supressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, VEGF, statines and the like, as well as their derivatives and analogs.

Other active agents or components of active agents may include, e.g., heparin, synthetic heparin analogs (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid (i.e. aspirin), ticlopidine, clopidogrel, abciximab, dextrans; corticosteroids such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs) such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics such as alkaloides and podophyllum toxins such as vinblastine, vincristine; alkylating agents such as nitrosoureas, nitrogen lost analogs; cytotoxic antibiotics such as daunorubicin, doxorubicin and other anthracyclines and related substances, bleomycin, mitomycin; antimetabolites such as folic acid analogs, purine analogs or pyrimidine analogs; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenytoin, tocainid; class Ic antiarrhythmics, e.g., propafenon, flecainid(acetate); class II antiarrhythmics beta-receptor blockers such as metoprolol, esmolol, propranolol, metoprolol, atenolol, oxprenolol; class III antiarrhythmics such as amiodarone, sotalol; class IV antiarrhythmics such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers such as prazosin, urapidil, doxazosin, bunazosin, terazosin, indoramin; vasodilatators such as dihydralazine, diisopropylamine dichloracetate, minoxidil, nitroprusside sodium; other antihypertensives such as indapamide, co-dergocrine mesylate, dihydroergotoxin methanessulfonate, cicletanin, bosentan, fludrocortisone; phosphodiesterase inhibitors such as milrinon, enoximon and antihypotensives such as in particular adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGFβ, PDGF, VEGF, bFGF, TNFα, NGF, GM-CSF, IGF-a, IL-1, IL-8, IL-6, growth hormone; as well as adhesive substances such as cyanoacrylates, beryllium, silica; and growth factors such as erythropoetin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particulary recombinant BMPs such as recombinant human BMP-2 (rhBMP-2), bisphosphonate (e.g., risedronate, pamidronate, ibandronate, zoledronic acid, clodronsäure, etidronsäure, alendronic acid, tiludronic acid), fluorides such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as in particular β-lactam antibiotics, e.g., β-lactamase-sensitive penicillins such as benzyl penicillins (penicillin G), phenoxymethylpenicillin (penicillin V); β-lactamase-resistent penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analog reverse-transcriptase inhibitors and derivatives) such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In preferred exemplary embodiments of the present invention, the active ingredient can be applied in the form of a solution, dispersion or suspension provided in a suitable solvent or solvent mixture, optionally with subsequent drying. Suitable solvents can include those mentioned herein above.

The medical devices provided according to exemplary embodiments of the present invention can be functionalized for therapeutic and/or diagnostic purposes as described, for example, in International Patent Publication No. WO 2004/105826 and U.S. Patent Publication No. 2005/0079201. For example, the functionalization of stents, orthopedic implants and special embodiments as described in these publications may also be applied to the medical devices described herein.

A medical device according to exemplary embodiments of the present invention can also be used in combination with living organisms in vivo or in vitro. For this purpose, the device can be contacted or incubated in vitro with living organisms, including cells, viral vectors or microorganisms, and then incubated under appropriate environmental conditions to promote growth of the living organism and/or ingrowth into the porous structure of the composite material. In an exemplary embodiment of the present invention, the medical device can be used as a support for the culturing of animal or plant cells and/or tissue, such as organ cells or tissue selected from human or animal skin, liver, bone, blood vessels, etc., or microorganisms, enzymes and the like, in vivo or in vitro. The device can be provided as a scaffold for tissue engineering, optionally in a living organism or in a bioreactor, for therapeutic or diagnostic purposes, or any combinations thereof. The medical devices as described herein may be used as three-dimensional tissue structures (e.g., scaffolds) to guide the organization, growth and/or differentiation of cells in a process of forming functional tissue. The functional tissue so produced may serve as a tissue substitute that may be used to replace malfunctioning organs or tissues, e.g., skin, liver, bone, blood vessels and the like, or portions thereof.

Average pore sizes of the composite materials provided in accordance with exemplary embodiments of the present invention may be determined by SEM (Scanning Electron Microscopy) techniques, adsorptive techniques such as gas adsorption or mercury intrusion porosimetry, or by chromatographic porosimetry. Porosity and specific surface areas may be determined by N₂ or He absorption techniques, e.g., using an exemplary BET technique. Particle sizes, including those of the reticulating agents, may be determined using a CIS Particle Analyzer (Ankersmid), a TOT technique (Time-Of-Transition), an X-ray powder diffraction technique, a laser diffraction technique, or a TEM (Transmission-Electron-Microscopy) technique. Average particle sizes in suspensions, emulsions or dispersions may also be determined using, e.g., dynamic light scattering techniques. Solids contents of liquid mixtures may be determined using, e.g., gravimetric techniques or humidity measurements.

Certain exemplary embodiments of the present invention will now be further described by way of the following non-limiting examples.

### EXAMPLE 1

A homogeneous dispersion of soot, lamp-black (Degussa, Germany) having a primary particle size of about 90 to 120 nm in a phenoxy resin (Beckopox® EP 401, Cytec) was prepared using the following exemplary procedure. First, a parent solution of methylethylketone (31 g), 3.1 g Beckopox® EP 401 and 0.4 g of glycerin (Sigma Aldrich) (a cross linker) was prepared. A soot paste was prepared using 1.65 g Lamp Black and 1.65 g of a dispersing additive (Disperbyk® 2150, solution of a block copolymer in 2-methoxy-1-methylethylacetate, Byk-Chemie, Germany), and adding a portion of the methylethylketone/Beckopox® EP 401 parent solution. Subsequently, the paste was converted into a dispersion by adding the remaining parent solution using a Pentraulik® dissolver for 15 minutes to obtain a homogeneous dispersion.

The dispersion was observed to contain a total solids content of about 3.5%, which was determined using a humidity measurement device (Sartorius MA 50). The particle size distribution in the dispersion was D50=150 nm, which was determined using a laser diffractometer (Horiba LB 550).

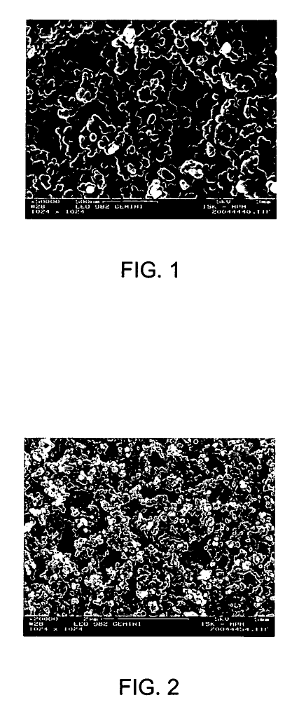

The dispersion was sprayed onto a steel substrate to an average areal weight of 4 g/m². Immediately after spraying, the layer was dried with hot air for 2 minutes. The sample was then thermally treated under a nitrogen atmosphere in a conventional tube furnace with a heating and cooling temperature rate of 1.33 K/min. The sample was heated to a maximum temperature, Tmax, of 280° C., held for 30 minutes at this temperature, and then cooled. The sample obtained from this process was examined with a scanning electron microscopy (SEM). FIG. 1 shows a 50,000× magnification of the resulting porous composite material layer having an average pore size of 100 to 200 nm.

### EXAMPLE 2

A homogeneous dispersion in a phenoxy resin was prepared as described in Example 1. However, 1.6 g of silica (Aerosil R972, Degussa, Germany) was used instead of soot. The dispersion was observed to have a total solids content of about 3.2%, and the average particle size distribution was D50=150 nm. The dispersion was sprayed onto a steel substrate to an average areal weight of 3.3 g/m² and dried with hot air for 2 minutes. A thermal treatment was then performed on this sample under the same conditions described in Example 1.

The resulting porous composite layer produced in this example is shown in the scanning electron microscopy image of FIG. 2 at 20,000× magnification. The sample was observed to have an average pore size of about 150 nm.

### EXAMPLE 3

A homogeneous dispersion of soot, lamp-black (Degussa, Germany) having a primary particle size of 90 to 120 nm, and fullerenes (Nanom Mix, FCC) and a phenoxy resin (Beckopox® EP 401, Cytec) was prepared using an exemplary procedure similar to the procedure described in Example 1. First, a parent solution of methylethylketone (31 g), 3.1 g Beckopox® EP 401 (resulting in a solids content of about 50%) and 0.4 g of glycerin (Sigma Aldrich) as a cross linker was prepared. A paste of the reticulating particles was prepared from 0.9 g lamp black, 0.75 g of the fullerene mixture and 1.65 g of a dispersing additive (Disperbyk 2150, Byk-Chemie, Germany), and a portion of the methylethylketone/Beckopox® EP 401 parent solution was added. Subsequently, the paste was converted into a dispersion by adding the remaining parent solution using a Pentraulik® dissolver for 15 minutes to obtain a homogeneous dispersion. The dispersion had a total solids content of about 3.6% (by weight), which was determined using a humidity measurement device (Sartorius MA 50). The particle size distribution in the dispersion was D50=1 μm, which was determined using a laser diffractometer (Horiba LB 550).

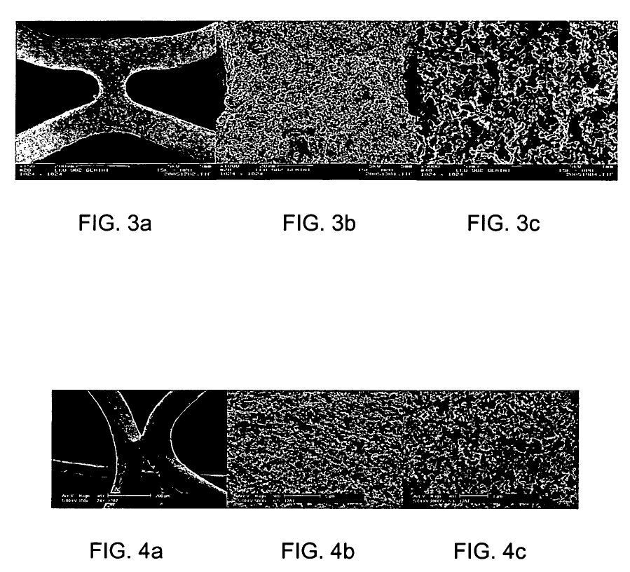

The dispersion was sprayed to an average areal weight of about 3.5 μg/mm² onto 10 commercially available coronary stents (KAON stent, 18.5 mm, Fortimedix Co. Netherlands) using a MediCoat® Stent-Coater (Sono-Tek, USA), and subsequently dried using a hot air fan (WAD 101, Weller Co. Germany) for 2 minutes. The coated stents were then thermally treated under a nitrogen atmosphere in a conventional tube furnace (Linn Co., Germany) using a heating and cooling temperature rate of 1.33 K/min. The coated stents were heated to maximum temperature, Tmax, of 280° C., held at this temperature for 30 minutes, and then cooled. The coatings were then cured for an additional 2 hours at 80° C. in a convection oven. The stents were then examined using a scanning electron microscope. FIGS. 3a-3c show exemplary SEM images of the porous, sponge-like composite coating layer obtained in this example at magnifications of 150×, 1,000× and 5,000×, respectively.

### EXAMPLE 4

One of the coated stents prepared in Example 3 was subjected to a further 30-minute treatment in an ultrasonic bath in acetone at 35° C. directly after the thermal treatment, and subsequently dried and cured for an additional 2 hours at 80° C. in a convection oven. FIGS. 4a-4c show exemplary SEM images of the porous, sponge-like coating layer obtained in this example at magnifications of 150×, 1,000× and 20,000×, respectively.

### EXAMPLE 5

A reticulated sponge-like, porous coating which may be used, e.g., for joint implants having a sponge-like scaffold structural interface to the bone tissue was prepared using the following procedure.

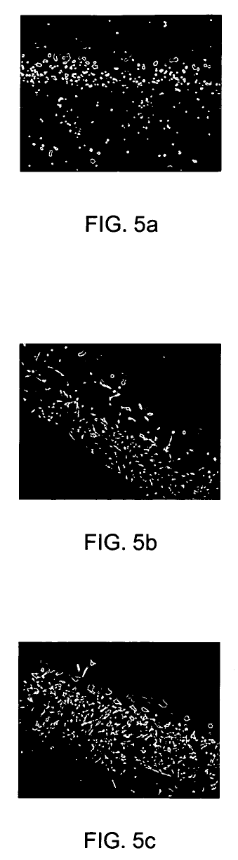

A homogeneous dispersion of soot, lamp-black (Degussa, Germany) having a primary particle size of 90 to 120 nm, fullerenes (Nanom Mix, FCC), and a phenoxy resin (Beckopox® EP 401, Cytec) was prepared as described in Example 3 above, using the same components and quantities thereof. 20 cylindrical samples of stainless steel 316L were dip coated with the dispersion and subsequently dried with a hot air fan (WAD 101, Weller Co. Germany) for 2 minutes. The coated samples were then thermally treated under a nitrogen atmosphere in a conventional tube furnace (Linn Co., Germany) using a heating and cooling temperature rate of 1.33 K/min. The coated samples were heated to maximum temperature, Tmax, of 280° C., held at this temperature for 30 minutes, and then cooled. The samples were then subjected to a 30-minute treatment in an ultrasonic bath in acetone at 35° C., directly after the 30 minute thermal treatment, and subsequently dried and cured for additional 2 hours at 80° C. in a convection oven. Then, the samples were sterilized in ethanol (98%) and individually incubated with 1 ml of an osteoblastic cell culture, comprising an average cell number of about 10⁶ cells, for 7 days. Previously, the cell culture was re-suspended in 1 ml Calcein AM and incubated for 30 minutes under CO₂, in order to perform a fluorescence microscopy vital staining. Samples were examined microscopically after incubation times of 120 minutes, 3 days, 5 days and 7 days. A regular adherence of the osteoblastic cells on the coated samples was observed after only 120 minutes, and then increased during 3, 5 and 7 days in an increasingly turbulent or trabecular orientation, respectively. FIGS. 5a-5c show microscopy images of the cell culture growing on the samples at incubation times of 120 minutes, 3 days and 5 days, respectively.

### EXAMPLE 6

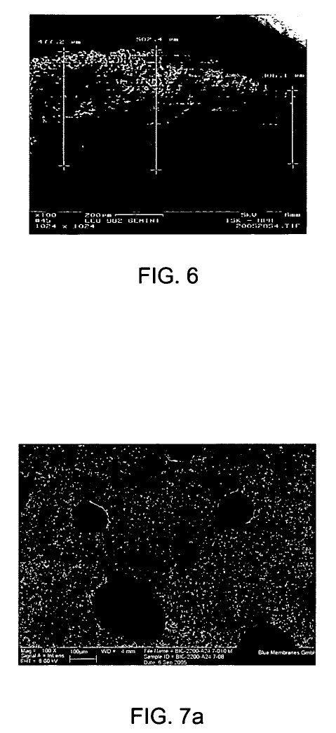

A porous, reticulated sponge-like composite was prepared which may be used, e.g., as a bone substitute material. 30 g of an epoxy-novolac resin (D.E.N. 438, Dow Chemical) was heated 80° C. while being stirred. 1 g of tantalum powder (HC Stark, Germany) having a medium particle size of about 3 μm, and 1 g of TiO₂ powder (Aeroxide P25, Degussa AG, Germany) having a medium particle size of about 25 nm, were dispersed in the stirred resin at 80° C. 2 ml of a cross linker solution consisting of 10 wt.-% phenylenediamine (Acros Organics), 40 wt.-% of diethylamine (Acros Organics), 1 wt.-% of dicyandiamide (Acros Organics), 9 wt.-% of ethylene amine (Acros Organics) and 40 wt.-% of Beckopox® EX651 (Cytec) was then added to the resin dispersion. The mixture was then poured into a mold and solidified in a convection oven at 80° C. for 24 hours. Thereafter, the molded padding was thermally treated in an air atmosphere at 200° C. A sample of this molded padding was cut into two parts, and the cut area was examined using an SEM. FIG. 6 shows an image of the cut surface of this sample at a magnification of 100×. The average pore size of this material was observed to be about 5 μm.

### EXAMPLE 7

1.87 g of a phenoxy resin (Beckopox EP 401 (Cytex) was placed in a mortar, and subsequently 0.635 g of tantalum particles having a medium particle size of about 3 μm (H.C. Stark) was added in portions, and the mixture was ground to form a substantially homogeneous paste.

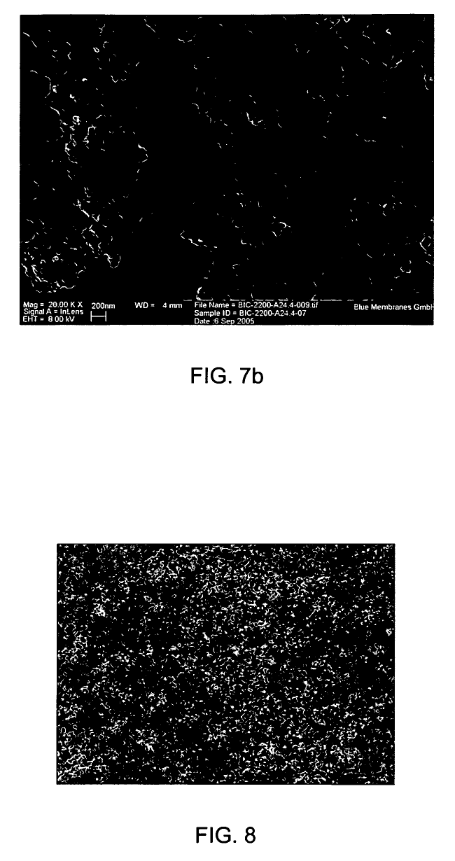

Separately, 0.626 g of titanium dioxide particles having a medium particle size of about 21 nm (Aeroxide P25, Degussa, Germany) was combined with 1.268 g of a dispersion aid (Dysperbyk P-104, Byk Chemie, Germany), ground to form a paste, and then diluted to form a dispersion by adding 4.567 g of methylethylketone. The dispersion was combined with the homogeneous paste of tantalum particles in the phenoxy resin, and 0.649 g of ethoxypropylacetate, 0.782 g of glycerin (a cross linker), 0.057 g of polyethylene particles having an average particle size of about 150 μm (Microscrub, Impag Company) and 0.126 g of polyethylene oxide (MW 300,000, Sigma Aldrich) were added. The resulting mixture was homogenized in a swing mill (Retsch) at 25 kHz for 2 minutes in the presence of 3 steel balls, each having a diameter of 1 cm. The resulting dispersion was dropped with a pipette onto a circular blank made of titanium and dried for 30 minutes in a conventional air convection oven at about 50° C. Subsequently, the sample was thermally treated at about 300° C. under a nitrogen atmosphere to cure the resin. The resulting material revealed microscopic pores having a size of about 100 to 200 μm, as shown in FIGS. 7a and 7b. Scanning electron microscopy analysis of the material revealed smaller pores having a reticulated, sponge-like structure in combination with the microscopic pores, resulting in a hierarchical porosity, as shown in FIG. 7a (100× magnification) and FIG. 7b (20,000× magnification).

### EXAMPLE 8

A tantalum-containing paste was produced as described above in Example 7. However, Dysperbyk® 180 (Byk Chemie, Germany) was used as the dispersion aid. This paste was then combined with a titanium dioxide-containing dispersion produced using the same components and quantities as that described in Example 7 above. Subsequently, 0.649 g of ethoxypropylacetate, 0.782 g glycerin (a cross linker), 0.057 g of polyethylene particles having a medium particle size of about 150 μm (Microscrub, available from Impag Company), and 0.126 g of polyethylene oxide (MW 300,000, Sigma Aldrich) were added as fillers or porogenes, respectively. The resulting mixture was homogenized in a swing mill (Retsch) at 25 kHz for 2 minutes with 3 steel balls, each having a diameter of 1 cm. The resulting dispersion was dropped with a pipette onto a circular blank made of titanium and dried for 30 minutes at 50° C. in a conventional air convection oven. The samples revealed a microscopically porous surface having a medium pore size of about 100 μm, as shown in FIG. 8a. FIG. 8b shows a 100× magnification of the image shown in FIG. 8a; which reveals the presence of macroscopic pores in a finely structured composite material having a microporous structure.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. In addition, all publications, patents and patent applications referenced herein are incorporated herein by reference in their entireties.

What is claimed is:

1. A medical device comprising:
a porous composite material which comprises at least one reticulating agent and at least one matrix material, wherein the at least one matrix material comprises at least one organic polymer.

2. The device of claim 1, wherein the at least one reticulating agent is embedded in the at least one matrix material.

3. The device of claim 1, wherein the composite material is capable of being obtained by:
providing a liquid mixture which further comprises the at least one reticulating agent and the at least one matrix material, and
solidifying the liquid mixture.

4. The device of claim 1, further comprising a coating which covers at least a portion of the device, wherein the coating comprises the porous composite material.

5. The device of claim 1, wherein the porous composite material has a reticulated structure.

6. The device of claim 1, wherein the at least one reticulating agent has a form of a plurality of particles.

7. The device of claim 6, wherein the particles are at least one of nanocrystalline particles or microcrystalline particles.

8. The device of claim 6, wherein the at least one reticulating agent comprises at least two particle size fractions, and wherein the particle size fractions differ in size from one another by a factor of at least 1.1.

9. The device of claim 8, wherein the particle size fractions differ in size from one another by a factor of at least 2.

10. The device of claim 1, wherein at least a portion of the reticulating agent has a form of at least one of a tube, a fiber or a wire.

11. The device of claim 10, where the at least one reticulating agent has a portion which includes a form of a particle.

12. The device of claim 1, wherein the at least one reticulating agent comprises an inorganic material.

13. The device of claim 12, wherein the at least one reticulating agent further comprises at least one organic material.

14. The device of claim 1, wherein the at least one reticulating agent comprises at least one of a magnetic metal, a superparamagnetic metal or a ferromagnetic metal, and wherein the reticulating agent further comprises at least one of iron, cobalt, nickel, manganese, an iron-platinum mixture, an iron-platinum alloy, a metal oxide, a magnetite of at least one of iron, cobalt, nickel or manganese, or a ferrite of at least one of iron, cobalt, nickel or manganese.

15. The device of claim 1, wherein the at least one reticulating agent comprises at least one of a particulate organic material or an organic fiber.

16. The device of claim 1, wherein the at least one organic polymer comprises at least one of a synthetic homopolymer of at least one of an aliphatic polyolefin or an aromatic polyolefin, a synthetic copolymer of at least one of an aliphatic polyolefin or an aromatic polyolefin, or a biopolymer.

17. The device of claim 1, further comprising an implant that is suitable for insertion into a human body or an animal body.

18. The device of claim 1, wherein the porous composite material further comprises at least one active agent, and wherein the at least one active agent comprises at least one of a biologically active agent, a therapeutically active agent, or an agent that is capable of being used for a diagnostic purpose.

19. The device of claim 18, wherein the device is capable of controllably releasing the at least one active agent in the presence of a physiologic fluid.

20. The device of claim 18, wherein at least one of the at least one reticulating agent or the at least one active agent comprises at least one of a marker, a contrast medium, or a radiopaque material.

21. The device of claim 1, wherein the porous composite material further comprises at least one further additive which includes at least one of fillers, surfactants, acids, bases, pore-forming agents, plasticizers, lubricants, or flame resistants.

22. The device of claim 1, wherein the at least one reticulating agent comprises a material that is capable of forming a network-type structure.

23. The device of claim 1, wherein the at least one reticulating agent comprises at least one of soot, a fullerene, a carbon fiber, silica, titanium dioxide, a metal particle, a tantalum particle, or a polyethylene particle; and wherein the organic polymer comprises at least one of an epoxy resin or a phenoxy resin.

24. The device of any one of claim 1, wherein the porous composite material has an average pore size of at least about 1 nm.

25. The device of any one of claim 1, wherein the porous composite material has an average pore size of at least about 10 nm.

26. The device of any one of claim 1, wherein the porous composite material has an average pore size of at least about 100 nm.

27. The device of claim 1, wherein the porous composite material has an average pore size between about 1 nm to about 400 μm.

28. The device of claim 1, wherein the porous composite material has an average pore size between about 500 nm to about 1000 μm.

29. The device of claim 1, wherein the porous composite material has an average porosity between about 30% and about 80%.
